(19) United States
(12) Patent Application Publication
(10) Pub. No.: US 2018/0000428 A1
(43) Pub. Date: Jan. 4, 2018

(54) Methods and Systems for Pre-Symptomatic Detection of Exposure to an Agent

(71) Applicant: Massachusetts Institute of Technology, Cambridge, MA (US)

(72) Inventors: Albert Swiston, Somerville, MA (US); Amanda Casale, Acton, MA (US); Shakti Davis, Arlington, MA (US); Mark Hernandez, Cambridge, MA (US); Lauren Milechin, Acton, MA (US)

(21) Appl. No.: 15/598,520

(22) Filed: May 18, 2017

Related U.S. Application Data
(60) Provisional application No. 62/337,964, filed on May 18, 2016.

(51) Int. Cl. A61B 5/00; G06F 19/00; A61B 5/0205; A61B 5/08; A61B 3/11; A61B 5/0488; A61B 5/0402; A61B 5/024; A61B 5/021; A61B 5/11; A61B 5/0476

(52) U.S. Cl. CPC A61B 5/7282; A61B 5/02055; A61B 5/7267; G06F 19/3431; A61B 5/082; A61B 3/112; A61B 5/0476; A61B 5/0402; A61B 5/024; A61B 5/021; A61B 5/112; A61B 5/4266; G06F 19/324; A61B 5/0488

(57) ABSTRACT

Systems and methods for predicting exposure to an agent. One or more features are extracted from physiological data. For each respective classifier, (i) the respective classifier is identified, wherein the respective classifier is trained using training data for a respective physiological state, (ii) the respective classifier is applied to the one or more features to obtain a classifier output that represents a likelihood of exposure, (iii) a respective first threshold is applied to the classifier output to determine a patient state classification, and (iv) the patient state classifications are aggregated across a number of time intervals to obtain an aggregate patient state classification for each classifier. The aggregate patient state classifications are combined across the plurality of classifiers to obtain a combined classification, and an indication that the patient has been exposed to the agent is provided when the combined classification exceeds a second threshold.

### CROSS REFERENCE TO RELATED APPLICATIONS

This application claims the benefit of U.S. Provisional Application No. 62/337,964, filed on May 18, 2016, which is hereby incorporated herein by reference in its entirety. This application is related to co-pending PCT Application No. ______ (Attorney Docket No. MIN-153-WO1) filed May 18, 2017, which is hereby incorporated herein by reference in its entirety.

### STATEMENT REGARDING FEDERALLY SPONSORED RESEARCH

This invention was made with Government support under Contract No. FA8721-05-C-0002 awarded by the U.S. Air Force. The Government has certain rights in the invention.

### TECHNICAL FIELD

In general, this disclosure relates to pre-symptomatic detection of exposure to a chemical or biological agent, and in particular, to systems and methods for pre-symptomatic detection of infection or intoxication using physiological data.

### BACKGROUND

Traditional biological infection or chemical intoxication detection occurs after agent exposure results in overt symptoms, and relies on specialized technology not appropriate for field use. In addition to characteristic clinical presentations, most infectious disease diagnosis is based upon identification of pathogen-specific molecular signatures (via culture, PCR/RT-PCR or sequencing for DNA or RNA, or immunocapture assays for antigen or antibody) in a relevant biological fluid. New approaches allowed by high-throughput sequencing have shown the promise of pre-symptomatic detection using genomic or transcriptional expression profiles in the host. However, these approaches suffer from often prohibitively steep logistic burdens and associated costs (cold chain storage, equipment requirements, extremely qualified operators, serial sampling). Indeed, most infections presented clinically are never definitively determined etiologically, much less serially sampled. Furthermore, molecular diagnostics are rarely used until patient self-reporting and presentation of overt clinical symptoms, such as fever. Past physiological signal based early infection detection work has been heavily focused on bacterial infection and largely centered upon higher time resolution analysis of body core temperature, advanced analyses of strongly-confounded signals such as heart rate variability, or social dynamics, or sensor data fusion from already symptomatic (febrile) viral-infected individuals. While progress has been made in developing techniques for signal-based early warning of bacterial infections and other critical illnesses in a hospital setting, there appear to be no efforts in extending these techniques to possibly life-threatening viral infections or other communicable pathogens.

### SUMMARY

Systems and methods are disclosed herein for predicting whether a patient has been exposed to an agent. For each respective time interval in a plurality of time intervals, physiological data regarding the patient that was recorded during the respective time interval is received. One or more features from the physiological data are extracted, wherein each feature is representative of the physiological data during the respective time interval. For each respective classifier in a plurality of classifiers, (i) the respective classifier is identified, wherein the respective classifier is trained using training data for a respective physiological state, (ii) the respective classifier is applied to the one or more features to obtain a classifier output that represents a likelihood that the patient has been exposed to the agent, (iii) a respective first threshold is applied to the classifier output to determine a patient state classification, and (iv) the patient state classifications are aggregated across a number of time intervals to obtain an aggregate patient state classification for each classifier. The aggregate patient state classifications are combined across the plurality of classifiers to obtain a combined classification, and an indication that the patient has been exposed to the agent is provided when the combined classification exceeds a second threshold.

In one embodiment, the plurality of classifiers includes a first classifier and a second classifier, the first classifier is trained using pre-fever training data, and the second classifier is trained using post-fever training data. The plurality of classifiers may further include a third classifier that is trained using training data following the pre-fever training data and preceding the post-fever training data. The pre-fever training data may be used to train the first classifier is recorded over a 24-hour period, and the post-fever training data may be used to train the second classifier is recorded over a 24-hour period.

In one embodiment, the respective first thresholds at (iii) are determined based on a desired probability of false alarm for each respective classifier.

In one embodiment, the second threshold is determined based on a performance metric of the system that is related to a probability of false alarm, a probability of detection, or early warning purity.

In one embodiment, the patient state classification in (iii) is a binary value indicative of a prediction by the respective classifier of whether the patient is exposed or not exposed, and the aggregating in (iv) includes summing across the binary values. The aggregating in (iv) may further include normalizing the summed binary values by the number of time intervals to obtain an averaged score for each respective classifier. The combining in (d) may include determining a maximum averaged score across the plurality of classifiers. The second threshold in (e) may be determined based on a ratio m/n, where n is the number of time intervals in (iv) and m is an integer greater than 0 and less than or equal to n.

In one embodiment, the physiological data solely includes an electrocardiogram signal obtained from a non-invasive wearable device on the patient. In another embodiment, the physiological data solely includes an electrocardiogram signal and a temperature signal obtained from at least one non-invasive wearable device on the patient. The one or more features may include solely heart rate and temperature.

In one embodiment, the agent is a first agent, and the training data includes data from subjects that were exposed to a second agent that is different from the first agent. In one embodiment, the patient is a human, and the training data includes data from non-human animal subjects.

In one embodiment, the extracting includes standardizing the physiological data such that the extracted one or more features are allowed to be compared across the respective time intervals.

In one embodiment, each extracted feature in is further representative of the physiological data during at least one time interval previous to the respective time interval.

### BRIEF DESCRIPTION OF THE DRAWINGS

The above and other features of the present disclosure, including its nature and its various advantages, will be more apparent upon consideration of the following detailed description, taken in conjunction with the accompanying drawings in which:

FIG. 1 is a block diagram of a classification system for determining a physiological state classification associated with physiological data, according to an illustrative implementation of the disclosure;

FIG. 2 is a block diagram of a training system for training a set of classifiers on physiological data, according to an illustrative implementation of the disclosure;

FIG. 3 is a block diagram of a testing system for testing a set of trained classifiers on physiological data, according to an illustrative implementation of the disclosure;

FIG. 4 is a block diagram of an application system for using trained and tested classifiers to determine a physiological state classification associated with physiological data, according to an illustrative implementation of the disclosure;

FIG. 5 is a block diagram of a computing device for performing any of the processes described herein, according to an illustrative implementation of the disclosure;

FIG. 6 is a flow diagram depicting a process, at the training stage, for training a set of classifiers on physiological data, according to an illustrative implementation of the disclosure;

FIG. 7 is a flow diagram depicting a process, at the application stage, for testing and using classifiers to determine a physiological state classification associated with physiological data and to provide a declaration of exposure, according to an illustrative implementation of the disclosure;

FIG. 8 is a flow diagram depicting a method for detection of exposure to an agent, according to an illustrative implementation of the disclosure;

FIG. 9 is a schematic of a probability of detection for current symptoms-based detection, an ideal signal, and a typical evolution of symptoms, according to an illustrative implementation of the disclosure;

FIGS. 10 and 11 are block diagrams of systems that predict whether a subject has been exposed to an agent, according to an illustrative implementation of the disclosure;

FIG. 12 is set of plots that depict the results of a data standardization process applied to temperature and heart rate data, and a typical evolution of symptoms, according to an illustrative implementation of the disclosure;

FIGS. 13 and 14 are sets of plots that depict exemplary detection and declaration results for example subjects, according to an illustrative implementation of the disclosure;

FIG. 15 is a set of plots that depict good performance of the exposure detection processes described herein when all features are considered, as well as when only ECG features are considered, according to an illustrative implementation of the disclosure; and

FIG. 16 is a set of plots that depict performance evaluation across different detection logic parameters m and n, according to an illustrative implementation of the disclosure.

### DETAILED DESCRIPTION

To provide an overall understanding of the systems and methods described herein, certain illustrative embodiments will now be described, including a system for pre-symptomatic detection of exposure to an agent using physiological data classifiers. However, it will be understood by one of ordinary skill in the art that the systems and methods described herein may be adapted and modified as is appropriate for the application being addressed and that the systems and methods described herein may be employed in other suitable applications, and that such other additions and modifications will not depart from the scope thereof. Generally, the computerized systems described herein may comprise one or more local or distributed engines, which include a processing device or devices, such as a computer, microprocessor, logic device or other device or processor that is configured with hardware, firmware, and software to carry out one or more of the computerized methods described herein.

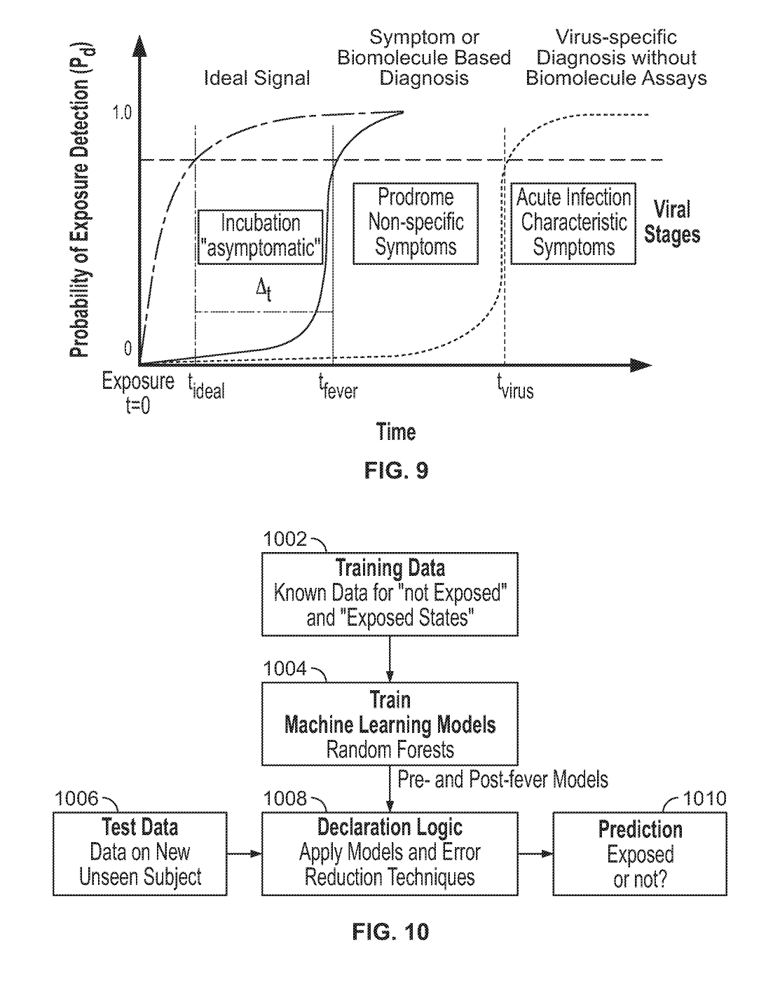

The disclosure describes, among other things, technical details of methods and systems for providing early warning of viral infections by using physiological monitoring before symptoms become apparent. The present disclosure relates to assessing pathogen exposure based solely on host physiological waveforms, in contrast to conventional diagnostics based on fever or biomolecules of the pathogen itself or the host's immune response. Early warning of pathogen exposure has many advantages: earlier patient care increases the probability of a positive prognosis and faster public health measure deployment, such as patient isolation and contact tracing, which reduces transmission. Following pathogen exposure, there exists an incubation phase where overt clinical symptoms are not yet present. This incubation phase can vary from days to years depending on the virus, and is reported to be 3-25 days for many hemorrhagic fevers and 2-4 days for Y. pestis. Following this incubation phase, the prodromal period is marked by non-specific symptoms such as fever, rash, loss of appetite, and hypersomnia. FIG. 9 presents a conceptual model of the probability of infection detection P_{d} during different post-exposure periods (incubation, prodrome, and virus-specific symptoms) for current specific and non-specific (i.e., symptoms-based) diagnostics. In particular, an ideal sensor and analysis system would be capable of detecting exposure for a given P_{d} (and probability of false alarm P_{fa}) soon after exposure and during the earliest moments of the incubation period (t_{ideal}), well before the non-specific symptoms of the prodrome (t_{fever}). Quantifiable abnormalities (versus a diurnal baseline, for instance) in high-resolution physiological waveforms, such as those from electrocardiography, hemodynamics, and temperature, before overt clinical signs could be a basis for the ideal signal, thereby providing advanced notice (the early warning time, Δₜ=t_{fever}−t_{ideal}) of on-coming pathogen-induced illness.

Implementing the type of early warning technique described herein could save lives of health care workers, military service members, patients, and other susceptible individuals. During the 2014 West Africa Ebola outbreak, for instance, health care workers at higher risk of viral exposure could have been monitored persistently for the earliest possible indications infection. More commonly, patients in post-operative or critical care units may be monitored for infection and treated well before clinical symptoms, viremia/bacteremia, or septic shock. In future, etiologically-specific iterations of this approach, knowledge of causative pathogens may inform very early therapeutic intervention. Furthermore, using very feature-limited datasets, such as those that could be collected using wearable sensor platforms, would enable the techniques described herein to be implemented in non-ideal clinical, athletic, and military environments. As used herein, the term “patient” may include humans as well as animals.

As used herein, the term “agent” includes a chemical substance, a biological substance, a viral pathogen, a bacterial pathogen, or any suitable combination thereof. Many of the examples described in the present disclosure include fever as a definitive indication of a symptom. In general, the present disclosure is not limited to fever as the only symptom, and the systems and methods described herein may be applied to other symptoms. For example, while fever is often a manifestation of exposure to biological substances, the corresponding symptoms for chemical substances may be highly varied. One important class of chemical substances are chemical nerve agents, which manifest as a cholinergic crisis and have characteristic symptoms that do not include fever. Many of the examples described herein use fever as a surrogate for obvious and overt symptoms, but those may include cholinergic crisis. The systems and methods of the present disclosure involve a high sensitivity and low specificity (that is, not informative of particular pathogens, exposure type, or species) processing and detection technique. The data is analyzed and anomalies are detected. The anomalies may indicate a pre-symptomatic infection, and may provide early warning about an infection well before an onset of fever. Quantitative analyses of the physiological data are conducted by extracting or determining several features, including summary statistics of the data, and performing classification, which may be done by random forest classifiers trained on respective post agent exposure time intervals, in an illustrative embodiment. Random forest classifiers are described herein by way of example only, and one of ordinary skill in the art will understand that other types of classifiers may be used without departing from the scope of the present disclosure, such as k-nearest neighbors classifiers and naive Bayes classifiers. In a first step, classifiers are trained on a set of physiological training data for which the patients' physiological states are known. A physiological state may correspond to the progression of an infection within a patient, the whether a patient was ever exposed to a agent, an alert state of the patient such as whether the patient is asleep or awake, a body position of the patient such as whether the patient is lying down, sitting, or standing, or any suitable classification that may be determined based on physiological data. In a second step, the classifiers are tested on a set of physiological testing data for their ability to detect infection in patients whose agent exposure time is known. In a third step, the classifiers are applied to a patient for which the physiological state is unknown. The classifiers will provide a detection indication when the number of classifiers predicting an infection in a given time interval exceeds a threshold, which is referred to as a detection. The classifiers will provide a declaration indication when the number of detection indications exceeds a threshold condition, which is referred to as a declaration. Detection and declaration indications may take any suitable format to indicate to users or elements of the present disclosure that the conditions for detection and declaration have been met. The systems and methods described herein demonstrate pre-symptomatic diagnostic potential, and may provide early warning about an infection well before an onset of fever. The time between the final declaration and the onset of fever is referred to herein as the “early warning time.”

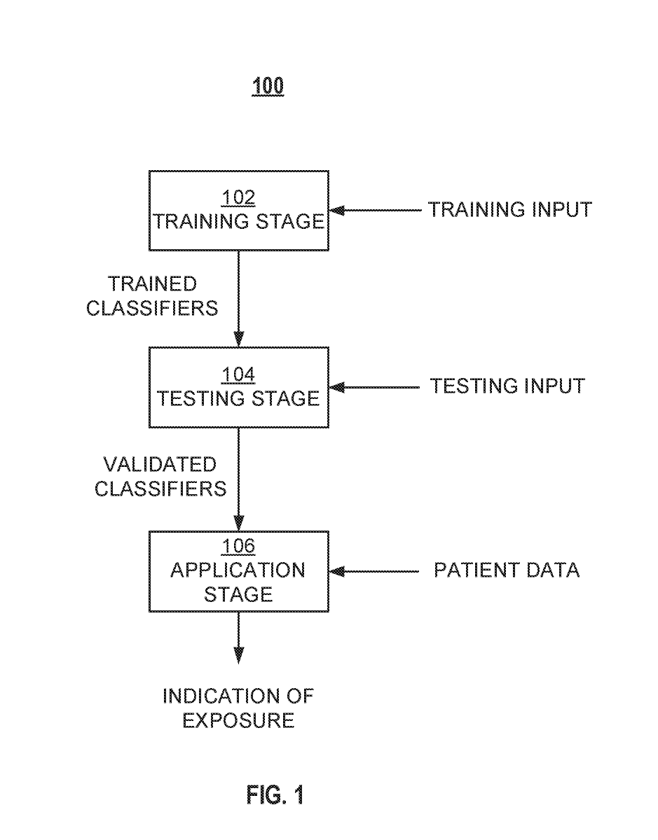

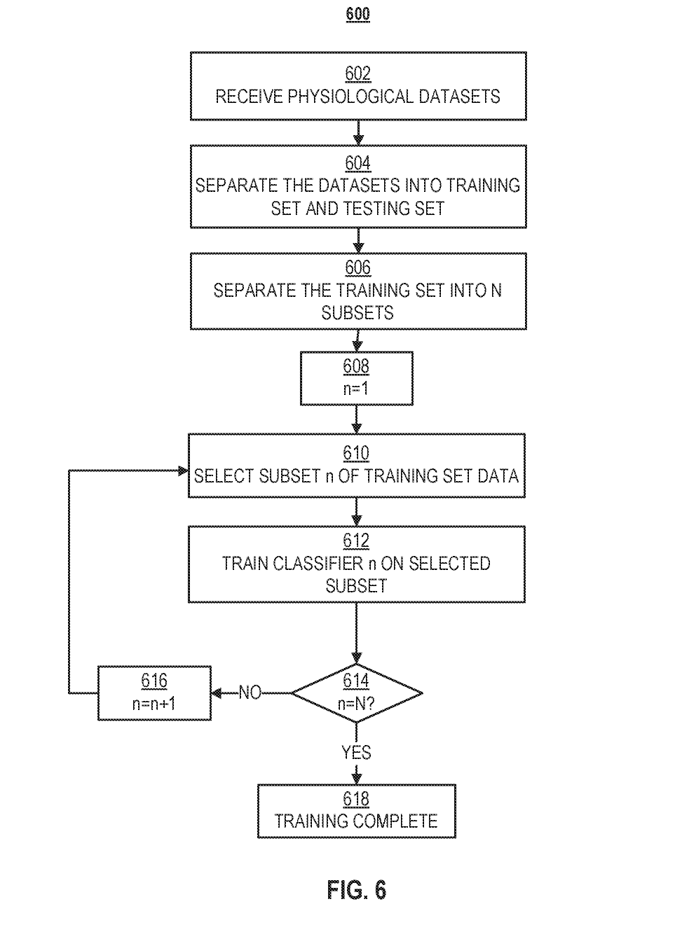

The systems and methods of the present disclosure may be described in more detail with reference to FIGS. 1-16. More particularly, an exemplary system for providing disease classification and its components are described with reference to FIGS. 1-5. The system may provide disease classification as described with reference to flow charts in FIGS. 6-8. In addition, results from an exemplary experiment are described with reference to FIGS. 9-16.

FIG. 1 is an illustrative block diagram of a classification system 100 for determining a physiological state classification associated with physiological data. The system 100 includes a training stage 102, a testing stage 104, and an application stage 106. Inputs to the system 100 include training input data to train a set of classifiers, testing input data to test the set of trained classifiers, and data recorded from a patient. The system 100 uses the trained and tested classifiers and the patient data to provide a predicted physiological state classification for the patient.

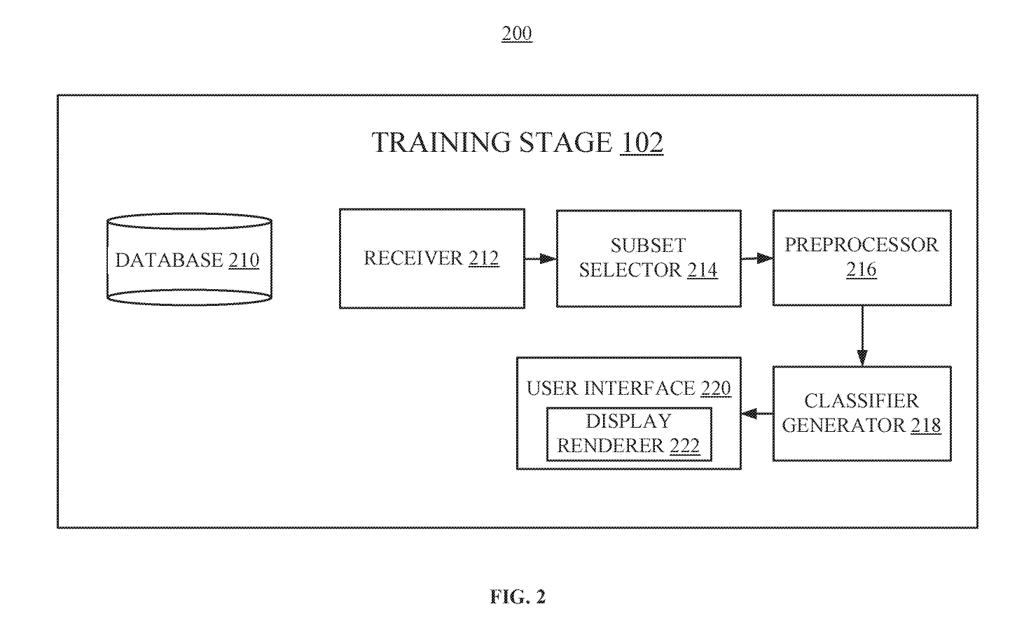

The training stage 102 receives a set of training input data and provides a set of trained classifiers to the testing stage 104. The set of training input data includes a set of training physiological data recorded from a first group of patients and a set of the times the patients were exposed to one or more agents. The components of the training stage 102 are described in detail in relation to FIG. 2, and the training stage 102 may operate on the training input data according to the method as described in relation to FIG. 6. In particular, the training stage 102 may select subsets of the training input data and train a classifier on each selected subset, for example by training each classifier on data from a respective time period, e.g. 24 hours, after agent exposure.

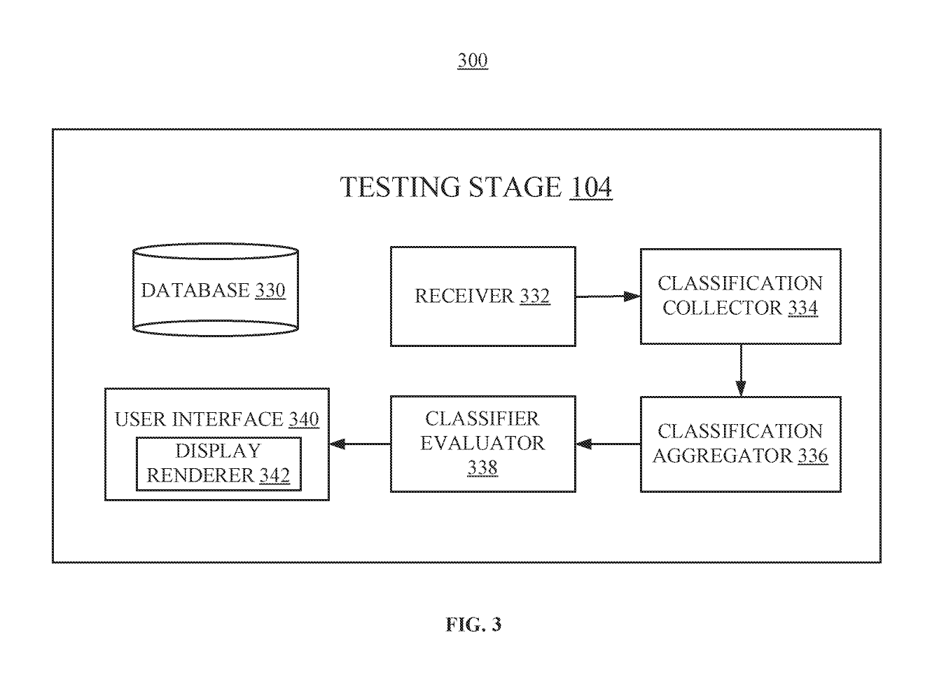

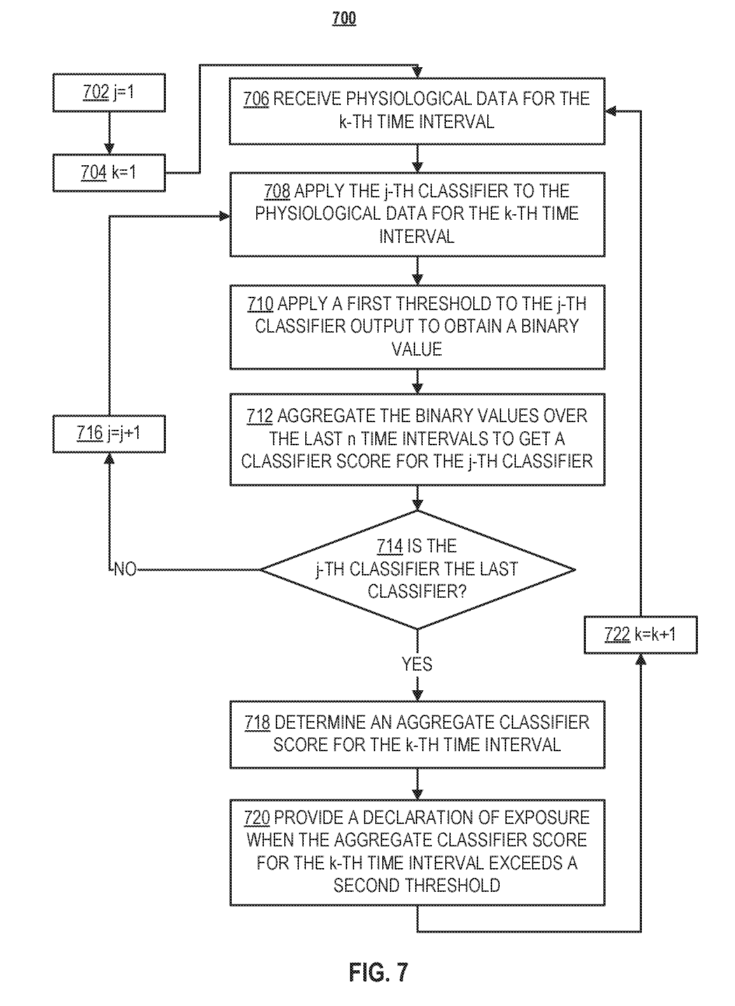

The testing stage 104 receives the set of trained classifiers from the training stage 102 and a set of testing input data. The set of testing input data includes a set of testing physiological data recorded from a second group of patients and a set of the times the patients were exposed to agents. The components of the testing stage 104 are described in detail in relation to FIG. 3, and the testing stage 104 may operate on the testing input data and the trained classifiers according to the method as described in relation to FIG. 7. In particular, the testing stage 104 may compare detection indications from the trained classifiers operating on the testing input data and compare the infection state classifications predicted by the detection indications to the corresponding set of actual physiological states from the second group of patients. If there is a sufficient match between the predicted and actual physiological states, the testing stage 104 validates the classifiers and provides the validated classifiers to the application stage 106.

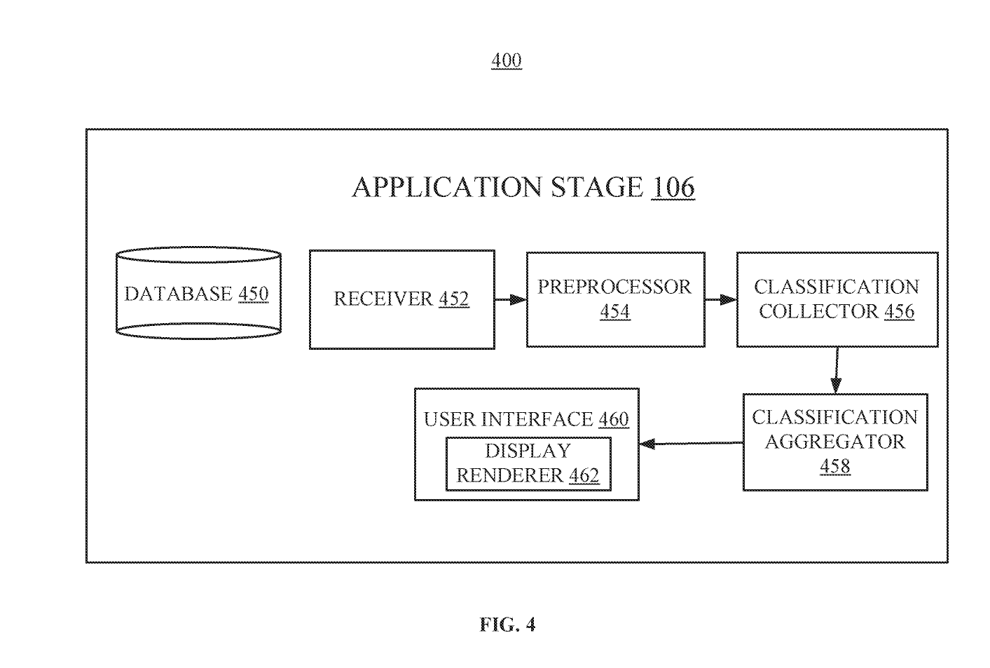

The application stage 106 receives the set of validated classifiers from the testing stage 104 and physiological data recorded from a patient, and the agent exposure of the patient may be unknown. The components of the application stage 106 are described in detail in relation to FIG. 4, and the application stage 106 may operate on the patient data and the validated classifiers according to the method as described in relation to FIG. 7. In particular, the application stage 106 may aggregate patient state classifications from the validated classifiers operating on the patient data to determine infection detection indications and declaration indications, which are defined in relation to FIG. 7. The indications of infection may be provided by the system 100 to a user such as a medical professional.

FIG. 2 is an illustrative block diagram of a training system 200 for training a set of classifiers on physiological data. The training stage 102 includes several components for executing the processes described herein. In particular, the training stage 102 includes a database 210, a receiver 212, a subset selector 214, a preprocessor 216, a classifier generator 218, and a user interface 220 that includes a display renderer 222. The training stage 102 may operate on training input data according to the method as described in relation to FIG. 6. The database 210 may be used to store any data related to training a set of classifiers as described herein.

The training stage 102 receives training input data over the receiver 212. The receiver 212 may provide an interface with a data source, which may transmit physiological training data and agent exposure data to the training stage 102. The physiological training data may be recorded from a first group of patients with respect to known agent exposure timing for the first group of patients and transmitted to the receiver 212. The physiological data may be recorded by any suitable means including implanted and wearable sensors. In particular, the training physiological data may include a number of physiological measurements, such as electrocardiogram data, pulmonary data, blood pressure data, temperature data, neurocognitive data (EEG), gait and ambulation measurements (actigraphy), speech data, muscle electrophysiology (EMG) data, pupil diameter measurements, sweat rate and salinity measurements, breath exhalate chemical analysis, and any other suitable physiological measurement.

After the training data are received, the subset selector 214 divides the training data into temporal subsets that include data recorded during specific time intervals, e.g. one time interval for each 12 hour period, 24 hour period, 36 hour period, or any other suitable time interval after agent exposure. In some implementations, the subset selector 214 selects only a portion, e.g. two thirds, one half, or any suitable portion, of the training data to be used in the training stage. The remaining training data may be reserved for use in the testing stage to cross validate the classifiers generated by the training stage.

The training data selected by the subset selector 214 is communicated to the preprocessor 216, which processes the training data to convert the data into a suitable form for performing classification. The preprocessor 216 may be used to eliminate short term fluctuations, eliminate diurnal rhythms, divide the data into time intervals, generate suitable summary statistics for each type of physiological data to be used as features for classification for each time interval, or any suitable combination thereof. In an exemplary implementation, the preprocessor 216 divides the training data into time intervals of a suitable length, e.g. 5, 10, 15, 30, 45, or 60 minutes, and calculates a mean value for each interval in order to eliminate short term fluctuations. To eliminate diurnal rhythms, each data point may be represented as a percent difference from the original point value and the mean value calculated for the respective time interval. The preprocessor 216 may then divide the training data into time intervals of the same or a different length, e.g. 15 minutes, 30 minutes, 60 minutes, or any suitable length of time, and extract suitable features for each time interval. For example, the preprocessor may calculate, for each time interval, a mean value, a standard deviation, and quartiles of the data values, which may be percent differences. These statistics may be used as the features that characterize the physiological data and may be calculated for any suitable physiological data, such as pulse data, ECG data, pulmonary data, blood pressure data, temperature data, and any other type of data that is physiologically recorded from the patient, and input to the patient state classifiers. These examples of physiological data are described by way of example only, and one of ordinary skill in the art will understand that other features of physiological data may be extracted without departing from the scope of the present disclosure. Moreover, a feature may be derived from a so-called “primary” feature, and two or more features may be correlated to one another if they are tied or related to the same primary feature. In one example, heart rate is tied to breath rate. In general, a magnitude of a periodicity modulation may be indicative of a health status of an individual. For example, healthy people may be associated with large modulation, while those with smaller modulation may be associated with heart disease, diabetes, or cancer. In addition, while the features are representative of the physiological data during the particular time interval that the physiological data was recorded, the features may also be indicative of the physiological data that was recorded during previous time intervals. The preprocessor 216 may also be configured to identify and remove outliers from the physiological data. The determination that a data point is an outlier, e.g. representative of a transient physiological anomaly, representative of a measurement error, or that is generally unsuitable for inclusion in the classification, may be made by the preprocessor 216.

The classifier generator 218 uses the features extracted by the preprocessor 216 to generate a patient state classifier for each time interval chosen by the subset selector 214. In some implementations, there is one classifier trained for each day, 12 hour interval, 36 hour interval, 48 hour interval, or any other suitable interval of data recorded after the patient was exposed to a agent as well as a baseline classifier that characterizes pre-exposure somatic function. In some implementations, the classifiers are random forest classifiers, each of which uses a set of decision trees to generate a final classification decision. In some implementations the random forests output a classification decision as well as a score indicating the proportion of trees in the forest whose individual output matched the forest classification or the proportion of trees whose classification indicates the presence of an infection. The random forest classifiers may be calibrated to output a patient state classification that indicates a prediction of the patient having been exposed to a agent only when the score exceeds a threshold, which may be determined by a target false prediction rate, sensitivity, specificity, or any suitable means. Additionally, the random forest classifiers may be used to determine the feature importance metrics of the input training features. The feature importance metric of a feature indicates how important a feature is to determining the final classification. The random forest classifiers may further output a list of the features that indicates the respective importance metric for each feature. The lists of predictively important features and any other suitable model output, including classifications and scores, can be output to a user via display renderer 222 or any suitable means.

In some implementations, the classifier generator 218 will train an intermediate classifier to identify the most predictive features, based on their feature importance metrics, e.g. those metrics that exceeds a threshold or the most predictive proportion of the features. A final classifier is then trained using the most predictive features. In some implementations, the user may specify which types of physiological data are used, e.g. classifiers that only use ECG data.

FIG. 3 is a block diagram of a testing system 300 for testing a set of trained classifiers on physiological data, according to an illustrative implementation of the disclosure. The testing stage 104 includes several components for executing the processes described herein. In particular, the testing stage 104 includes a database 330, a receiver 332, a classification collector 334, a classification aggregator 336, a classifier evaluator 338, and a user interface 340 including a display renderer 342. The testing stage 104 may operate on testing input data and a set of trained classifiers according to the method described in relation to FIG. 7. The database 330 may be used to store any data related to testing a set of classifiers as described herein.

The testing stage 104 receives testing input data and a set of trained classifiers over the receiver 332. The receiver 332 may provide an interface with a data source, which may transmit testing physiological data and corresponding agent exposure data to the testing stage 204. The testing physiological data may be recorded from a second group of patients (i.e., which may be different from the first group of patients making up the set of testing physiological data), and the agent exposure of the second group of patients may be known and transmitted to the receiver 332. In some implementations, the second group of patients is a portion of the testing data that was set aside during the training stage 102. Patient data set aside during the training stage 102 is not used to train the classifiers and can, therefore, be used to cross validate the classifiers. The patients within and across the first and second groups may not be infected with the same disease. Patients used for cross validation may not be infected with any disease. The receiver 332 may also form an interface with the training stage 102 to receive a set of trained classifiers from the training stage 102. In particular, each trained classifier in the set of trained classifiers may be trained on physiological data from a specific post agent exposure time interval.

After the testing data and the set of classifiers are received, the classification collector 334 collects classifications from the trained classifiers based on the physiological record from each patient in the second group of patients. The classifications correspond to candidate physiological state classifications that are output for a given time interval, e.g. 15 minutes, 30 minutes, or 1 hour, based on the likelihood of infection determined by each trained classifier. In some implementations, for each patient record in the set of testing physiological data and each time interval, the classification collector 334 determines whether the number of patient state classifications indicating infection meets or exceeds a threshold (e.g. a threshold level of 1 out of 6 classifiers or 2 out of 7 classifiers) and outputs an infection detection indication.

After the classifications for a time interval have been collected, the classification aggregator 336 aggregates the classifications. The classification aggregator 336 combines the classifications and detection indications from each time interval for a patient. When the number of infection detection indications in a certain number of recent time intervals exceeds a threshold, the classification aggregator 336 outputs an indication that the patient is ill, a declaration indication.

After the classifications are aggregated, the classifier evaluator 338 performs a validation of the classifiers. In particular, the classifier evaluator 338 compares the infection detections and declarations to the known physiological states of the second group of patients to determine a level of accuracy of the classifiers and to compare the declaration of illness to the onset of febrile symptoms. For example, the classifier evaluator 338 may determine that the classifiers are validated if the number of correctly declared illnesses exceeds a threshold or if the diagnoses are being made sufficiently close to agent exposure. The threshold may be a fixed number or a percentage and may be provided by a user over the user interface 340. If the classifier evaluator 338 determines that the trained classifiers are invalid, the testing stage 104 may provide an instruction to the training stage 102 to repeat the training process (e.g. trying a different set of features, a different number of classifiers, or a change in any other suitable parameter in the training process). For example, the testing stage 104 may return the rejected classifiers to the training stage 202. The rejected classifiers may be retrained using the most predictive features identified in the rejected classifier, based on their feature importance metrics, e.g. those metrics that exceeds a threshold or the most predictive proportion of the features. A new classifier is then trained using the most predictive features. These steps may be repeated until a set of classifiers is identified that satisfies the criterion required by the classifier evaluator 338. The testing stage 104 then provides the validated set of classifiers to the application stage 206.

FIG. 4 is a block diagram of an application system 400 for using trained and tested classifiers to determine a physiological state classification associated with physiological data, according to an illustrative implementation of the disclosure. The application stage 106 includes several components for executing the processes described herein. In particular, the application stage 106 includes a database 450, a receiver 452, a preprocessor 454, a classification collector 456, a classification aggregator 458, and a user interface 460 including a display renderer 462. The testing stage 104 may operate on testing input data and a set of trained classifiers according to the method described in relation to FIG. 7. The database 450 may be used to store any data related to testing a set of classifiers as described herein.

The application stage 106 receives a set of trained classifiers over the receiver 452. The receiver 452 may provide an interface with a data source, which transmits physiological data related to a patient to the application stage 106. The physiological data may be recorded from a patient that was not included in the training or testing groups of patients, and the agent exposure of the patient may be unknown. The recording may be done using high resolution monitors, surgically implanted monitors, wearable monitors, or any suitable physiological monitor. The receiver 452 may also form an interface with the training stage 102 to receive a set of trained classifiers from the training stage 102. In particular, each trained classifier in the set of trained classifiers may be trained on physiological data from a specific post agent exposure time interval.

Patient physiological data communicated to the receiver 452 is communicated to preprocessor 454, which processes the training data to convert the data into a suitable form for performing classification. The preprocessor 454 may be used to eliminate short term fluctuations, eliminate diurnal rhythms, divide the data into time intervals, generate suitable summary statistics for each type of physiological data to be used as features for classification for each time interval, or any suitable combination thereof. In an exemplary implementation, the preprocessor 454 divides the training data into time intervals of a suitable length, e.g. 5, 10, 15, 30, 45, or 60 minutes, and calculates a mean value for each interval in order to eliminate short term fluctuations. To eliminate diurnal rhythms, each data point may be represented as a percent difference from the original point value and the mean value calculated for the respective time interval. The preprocessor 454 may then divide the training data into time intervals of the same or a different length, e.g. 15 minutes, 30 minutes, 60 minutes, or any suitable length of time, and extract suitable features for each interval. For example, the preprocessor may calculate, for each time interval, a mean value, a standard deviation, and quartiles of the data values, which may be percent differences. These statistics may be used as the features that characterize the physiological data and may be calculated for any suitable physiological data, such as pulse data, ECG data, pulmonary data, blood pressure data, and temperature data, and input to the patient state classifiers.

In some embodiments, the preprocessor 454 standardizes the physiological data by subtracting the mean value and normalizing the difference by a standard deviation of the data. Details about a specific example of how the standardization is performed are described in relation to Experiment 1 below. These examples of physiological data are described by way of example only, and one of ordinary skill in the art will understand that other features of physiological data may be extracted without departing from the scope of the present disclosure. The preprocessor 454 may also be configured to identify and remove outliers from the physiological data. The determination that a data point is an outlier, e.g. representative of a transient physiological anomaly, representative of a measurement error, or that is generally unsuitable for inclusion in the classification, may be made by the preprocessor 454.

After the set of classifiers are received and as the physiological data is received and preprocessed, the classification collector 456 collects classifications from the set of trained classifiers based on the physiological data from the patient. The classifications correspond to candidate physiological state classifications that are output for a given time interval, e.g. 2 minutes, 5 minutes, 15 minutes, 30 minutes, or 1 hour, based on the likelihood of infection determined by each trained classifier. This time interval may be based on an expected speed of infection or intoxication. For example, when analyzing a likelihood of a chemical exposure, a time interval of 2 minutes may be used. In some implementations, the patient's physiological data is streamed to the receiver 452 in real time. In some implementations, the patient's physiological data is downloaded from a storage medium to the receiver 452 or database 450. In some implementations, for each time interval, the classification collector 456 determines whether the number of patient state classifications indicating infection meets or exceeds a threshold (e.g. a threshold level of 1 out of 6 classifiers or 2 out of 7 classifiers) and outputs an infection detection indication. In some implementations, the classification collector 456 applies each classifier in the set of classifiers to the same time interval. In some implementations, the classification applies each classifier to respective time intervals that are spaced apart by an amount equal to the length of the time period on which each classifier was trained. For example, if the classifiers were trained on 24 hour periods of post exposure data, then the classification collector 456 applies the classifiers to time intervals that are 24 hours apart, and the classification collector 456 applies this process once for each classifier in order to position each classifier as the most recent, since the time of agent exposure is unknown. This process can allow for early detection of infection as well as an estimated time of exposure.

After the classifications for a time interval have been collected, the classification aggregator 458 aggregates the classifications. The classification aggregator 458 combines the classifications and detection indications from each time interval for a patient. When the number of infection detection indications in a certain number of recent time intervals exceeds a threshold, the classification aggregator 458 outputs an indication that the patient is ill. This may be referred to herein as a declaration indication, which may be displayed to a clinician via user interface 460, display renderer 462, or any suitable means.

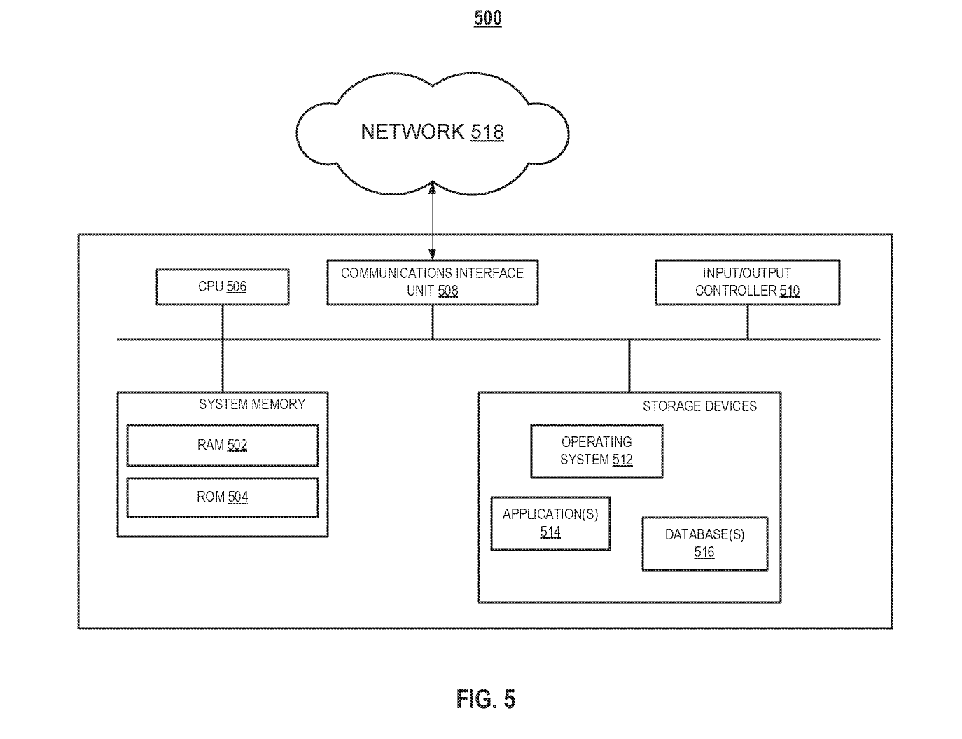

FIG. 5 is a block diagram of a computing device for performing any of the processes described herein, according to an illustrative embodiment. Each of the components of these systems may be implemented on one or more computing devices 500. In certain aspects, a plurality of the components of these systems may be included within one computing device 500. In certain implementations, a component and a storage device may be implemented across several computing devices 500.

The computing device 500 comprises at least one communications interface unit, an input/output controller 510, system memory, and one or more data storage devices. The system memory includes at least one random access memory (RAM 502) and at least one read-only memory (ROM 504). All of these elements are in communication with a central processing unit (CPU 506) to facilitate the operation of the computing device 500. The computing device 500 may be configured in many different ways. For example, the computing device 500 may be a conventional standalone computer or, alternatively, the functions of computing device 500 may be distributed across multiple computer systems and architectures. In FIG. 5, the computing device 500 is linked, via network or local network, to other servers or systems.

The computing device 500 may be configured in a distributed architecture, wherein databases and processors are housed in separate units or locations. Some units perform primary processing functions and contain at a minimum a general controller or a processor and a system memory. In distributed architecture implementations, each of these units may be attached via the communications interface unit 508 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM and TCP/IP.

The CPU 506 comprises a processor, such as one or more conventional microprocessors and one or more supplementary co-processors such as math co-processors for offloading workload from the CPU 506. The CPU 506 is in communication with the communications interface unit 508 and the input/output controller 510, through which the CPU 506 communicates with other devices such as other servers, user terminals, or devices. The communications interface unit 508 and the input/output controller 510 may include multiple communication channels for simultaneous communication with, for example, other processors, servers or client terminals in the network 518.

The CPU 506 is also in communication with the data storage device. The data storage device may comprise an appropriate combination of magnetic, optical or semiconductor memory, and may include, for example, RAM 502, ROM 504, flash drive, an optical disc such as a compact disc or a hard disk or drive. The CPU 506 and the data storage device each may be, for example, located entirely within a single computer or other computing device; or connected to each other by a communication medium, such as a USB port, serial port cable, a coaxial cable, an Ethernet cable, a telephone line, a radio frequency transceiver or other similar wireless or wired medium or combination of the foregoing. For example, the CPU 506 may be connected to the data storage device via the communications interface unit 508. The CPU 506 may be configured to perform one or more particular processing functions.

The data storage device may store, for example, (i) an operating system 512 for the computing device 500; (ii) one or more applications 514 (e.g., computer program code or a computer program product) adapted to direct the CPU 506 in accordance with the systems and methods described here, and particularly in accordance with the processes described in detail with regard to the CPU 506; or (iii) database(s) 516 adapted to store information that may be utilized to store information required by the program.

The operating system 512 and applications 514 may be stored, for example, in a compressed, an un-compiled and an encrypted format, and may include computer program code. The instructions of the program may be read into a main memory of the processor from a computer-readable medium other than the data storage device, such as from the ROM 504 or from the RAM 502. While execution of sequences of instructions in the program causes the CPU 506 to perform the process steps described herein, hard-wired circuitry may be used in place of, or in combination with, software instructions for implementation of the processes of the present disclosure. Thus, the systems and methods described are not limited to any specific combination of hardware and software.

Suitable computer program code may be provided for performing one or more functions in relation to performing classification of physiological states based on physiological data as described herein. The program also may include program elements such as an operating system 512, a database management system and “device drivers” that allow the processor to interface with computer peripheral devices (e.g., a video display, a keyboard, a computer mouse, etc.) via the input/output controller 510.

The term “computer-readable medium” as used herein refers to any non-transitory medium that provides or participates in providing instructions to the processor of the computing device 500 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 506 (or any other processor of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 500 (e.g., a server) can receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information.

The systems shown in FIGS. 1-5 may allow for pre-fever infection detection as described with reference to flowcharts in FIGS. 6-8. In particular, the training stage 102 may use the method shown in FIG. 6 to train a set of classifiers on a set of physiological training data. After the set of classifiers are trained, the testing stage may use the method shown in FIG. 7 to validate the set of trained classifiers. Finally, the application stage may use the method shown in FIG. 7 to apply the validated classifiers to a patient's physiological data to identify a predicted physiological state of the patient.

FIG. 6 is a flow diagram depicting a process, at the training stage, for training a set of classifiers on physiological data, according to an illustrative implementation of the disclosure. The method 600 includes the steps of receiving physiological datasets (step 602), separating the dataset into a training set and a testing set (step 604), separating the training set into N subsets (step 606), and initializing an iteration parameter n to one (step 606). The n-th subset of the training set data is selected (step 610), and an n-th classifier is trained on the selected subset (step 612). Steps 610 and 612 are repeated until the desired number of classifiers (i.e., N), which may be configured by the user, have been trained.

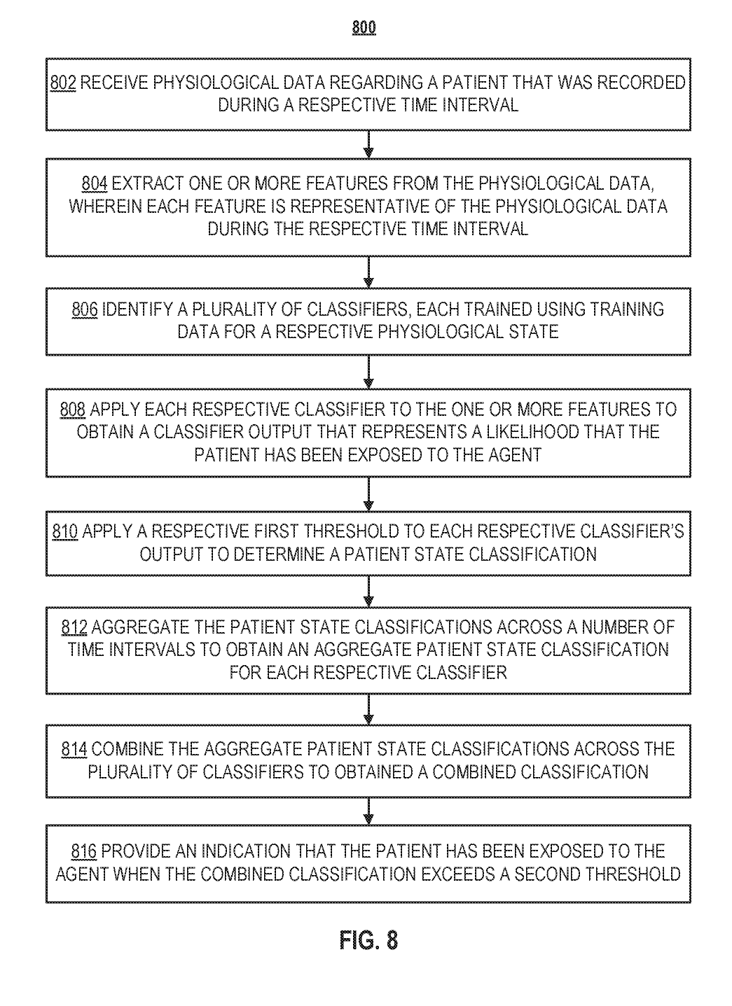

At step 602, physiological datasets are received, for which agent exposure times are known. At step 604, the received datasets are separated into a training set and a testing set. The training set is used to develop the classifiers and is provided as input to the training stage 102. The testing set is used to assess the performance of the resulting classifiers and is provided as input to the testing stage 104. An example method of assessing the performance of the classifiers in the testing stage 104 is described in relation to FIG. 8.

At step 606, the received datasets are divided into N subsets, e.g. by subset selector 214. Each subset of the training data includes data recorded during specific time intervals, e.g. one time interval for each 12 hour period, 24 hour period, 36 hour period, or any other suitable time interval after agent exposure. At step 608, the iteration parameter n is initialized to one. The iteration parameter n is representative of a selected subset of the training set.

At step 610, the subset selector 214 selects an n-th subset of the training set data. Optionally, the training set data may be processed by the preprocessor 216 (e.g., to get the training set data into a suitable form). These processes are described in more detail in relation to FIG. 3.

At step 612, the n-th classifier is trained on the corresponding subset. In some implementations, there is one classifier trained for each day, 12 hour period, 36 hour period, or 48 hour period of data recorded after the patient was exposed to an agent as well as a baseline classifier that characterizes pre-exposure somatic function. In some implementations, the classifiers are random forest classifiers, each of which uses a set of decision trees to generate a final classification decision.

At decision block 614, it is determined whether the iteration parameter n equals the desired total number of subsets N. In an exemplary implementation, N is set to 7, and there are seven classifiers each trained on a respective day of a week of post exposure data. In an example, the total number of subsets N may be set to a larger number (such as 10, 25, 50, 100, for example), and the results may be analyzed until a plateau in performance is reached. Using a larger value for N generally involves more computation, so it may be desirable to set N to a value that is large enough to achieve a desired performance but small enough to be computationally efficient. In one example, N may be set to 50 in order to achieve a plateau in performance while being computationally efficient. If n does not equal N, the iteration parameter n is incremented at step 616 and the process 600 returns to step 610 to select the next subset of training set data. When iteration parameter n has reached its final value N, training is complete at step 618. In particular, as a result of the training, N classifiers have been generated. The classifiers may be different because they were tuned for optimal performance on different subsets of the training set records, though each classifier resulted from the same mathematical or computational structure.

In some implementations, the number N of classifiers is three: one baseline pre-exposure classifier that is trained on pre-exposure data obtained from the same patient or a population of patients, one post-exposure and pre-symptomatic classifier that is trained on data that was recorded after exposure to an agent but before the patient exhibited symptoms of infection or intoxication, and one post-exposure and post-symptomatic classifier that is trained on data that was recorded after exposure to the agent and after the patient began to exhibit symptoms of infection or intoxication. Rather than using a different classifier for each fixed post-exposure time interval, this method of using just three classifiers defined based on exposure time and time of symptom(s) arising may be advantageous because of its simplicity.

In some implementations, the number N of classifiers is two: one post-exposure and pre-symptomatic classifier that is trained on data that was recorded after exposure to an agent but before the patient exhibited symptoms of infection or intoxication, and one post-exposure and post-symptomatic classifier that is trained on data that was recorded after exposure to the agent and after the patient began to exhibit symptoms of infection or intoxication.

FIG. 7 is a flow diagram depicting a process, at the application stage or testing stage, for testing and using classifiers to determine an exposure status associated with physiological data, according to an illustrative implementation of the disclosure. The method 700 includes the steps of initializing a first iteration parameter j and a second iteration parameter (steps 702 and 704), receiving physiological data for the k-th time interval (step 706), applying the j-th classifier to the physiological data for the k-th time interval (step 708), applying a first threshold for the j-th classifier output to obtain a set of binary values (step 710), and aggregating the binary values over the last n time intervals to get a classifier score for the j-th classifier (step 712). These steps are repeated until the last classifier is considered. Then, an aggregate classifier score for the k-th time interval is determined (step 718), and a declaration of exposure is provided when the aggregate classifier score for the k-th time interval exceeds a second threshold (step 720). These steps are repeated for different time intervals.

At step 702, the first iteration parameter j is initialized to 1, and at step 704, the second iteration parameter k is initialized to 1. At step 706, physiological data for the k-th time interval is received from a patient. The physiological data may be preprocessed as discussed in relation to FIG. 4.

At step 708, the j-th classifier is applied to the physiological data for the k-th time interval. Specifically, a set of trained classifiers (e.g. those trained in relation to FIG. 6) provide a classifier output based on one or more features that are extracted from the physiological data. The classifier output is a score that ranges from 0 to 1 and is indicative of a predicted likelihood of exposure, based on the respective classifier. In some implementations, the classifiers are random forest classifiers that are trained on different time interval relative to exposure. The classifiers may give different levels of significance to different features of the physiological data.

At step 710, a first threshold is applied to the j-th classifier output to obtain a binary value. As is explained in U.S. patent application Ser. No. 15/212,769, which is hereby incorporated herein by reference in its entirety, each classifier may be associated with a particular maximum probability of false alarm, by setting the threshold required for a classification indicating exposure. In some implementations, the threshold determines the number or proportion of decision trees in a random forest that are required to vote for a classification indicating exposure in order for the entire forest to output the classification. Thresholds may be set individually for each classifier. For each classifier, a probability of false alarm can be calculated by using baseline, pre-exposure physiological data to check for false positives for every threshold. The threshold can then be set sufficiently high to limit the probability of false alarm, such as to 0.001%, 0.01%, 0.1%, 0.5%, 1%, 5%, or any other suitable percentage.

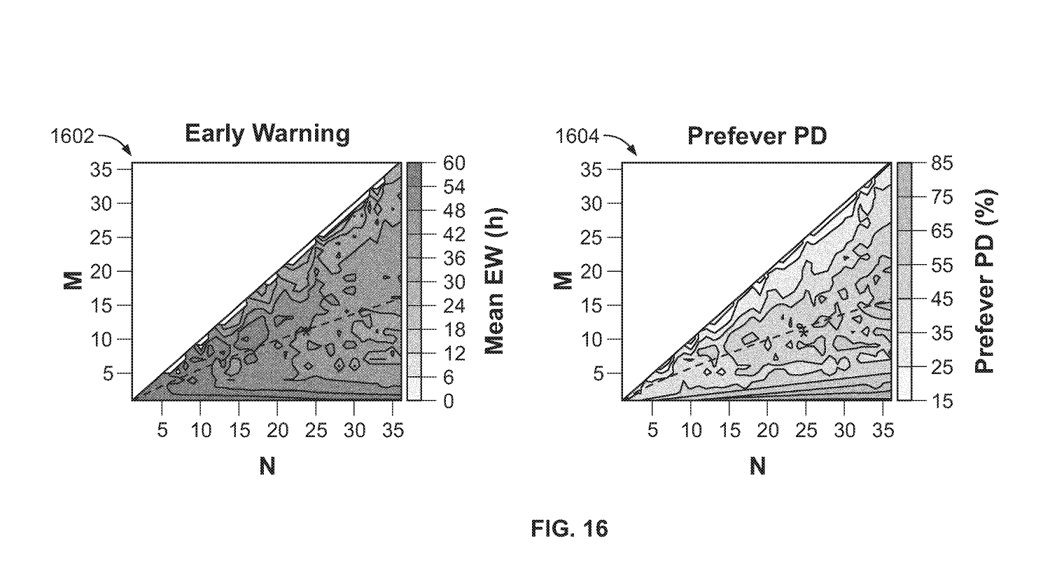

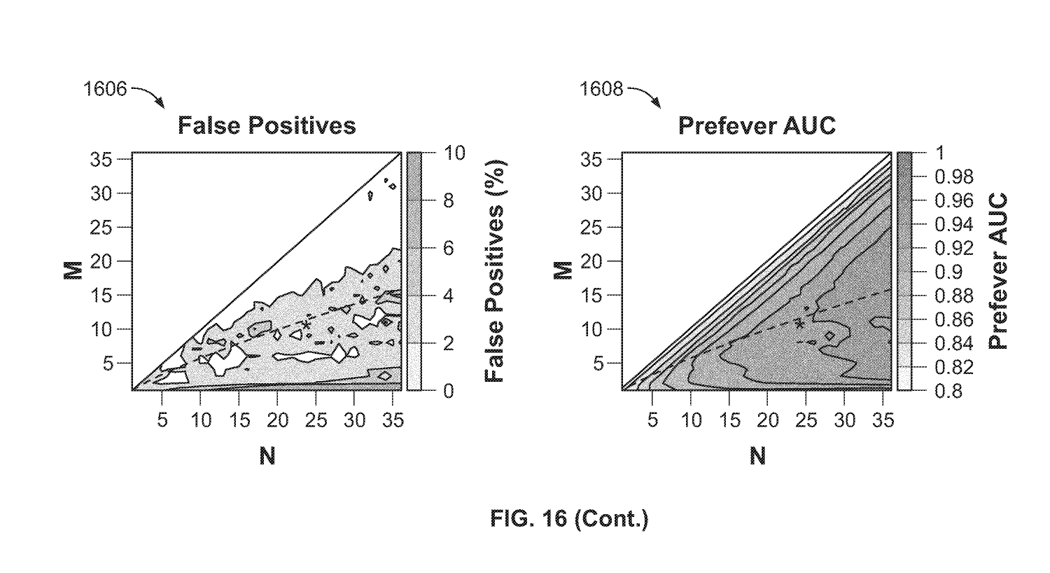

At step 712, the binary values obtained at step 710 are aggregated over the last n time intervals to obtain a classifier score for the j-th classifier. The aggregation at step 712 may include a binary integration, where the binary values are summed over the last n time intervals. As is explained in detail in relation to Experiment 1 and FIG. 16, the value for n may be selected to include a sufficient number of time intervals. In one example, the value for n is related to a system latency, or a shortest possible time between the first detections and the final declaration (for the specified probability of false alarm, or P_{fa}) that is associated with a higher confidence than the first detections. In some embodiments, the value for n is selected based on a specific type of infection and/or a specific consequence. For example, in sepsis, a value for n that results in a 12 hour latency (e.g., n=24 when the time intervals are each 30 minutes long) may be too long, as the patient may die before the system outputs a declaration of exposure. For certain viral infections that may take around 3 to 4 days between exposure to the virus and fever, a 12 hour latency (e.g., n=24 when the time intervals are each 30 minutes long) may be sufficient for the time course of such viral infection.

If the j-th classifier is not the last classifier (decision block 714), the process 700 proceeds to step 716 to increment the iteration parameter j, and then proceeds to repeat steps 708, 710, and 712 for the j-th classifier. When all the classifiers have been used (decision block 714), the process 700 proceeds to step 718 to determine an aggregate classifier score for the k-th time interval. As is explained in detail in relation to Experiment 1, the aggregate classifier score may correspond to the maximum classifier score across all the classifiers. In some embodiments, the aggregate classifier score may correspond to another statistic related to the classifier scores. For example, the aggregate classifier score may correspond to a statistic such as a mean or a rolling average. In general, the aggregate classifier score may correspond to some metric that includes an integration of a function over time, in which recent values may be more heavily weighted than older values.

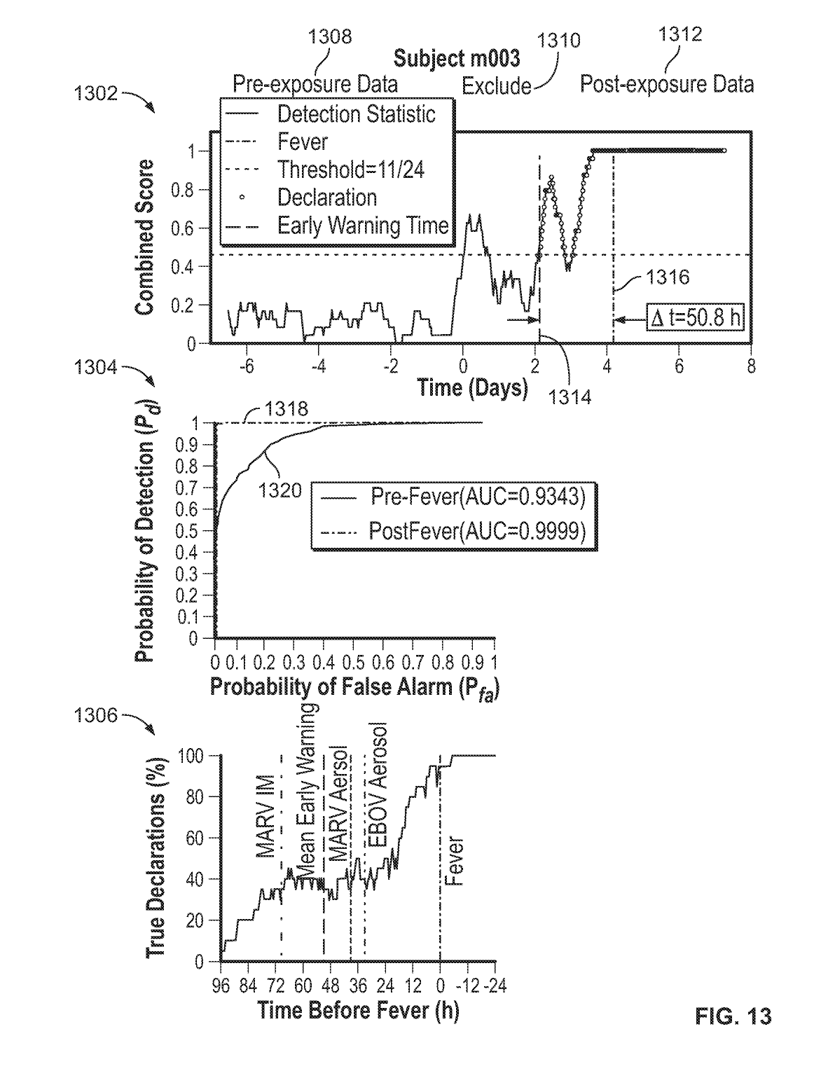

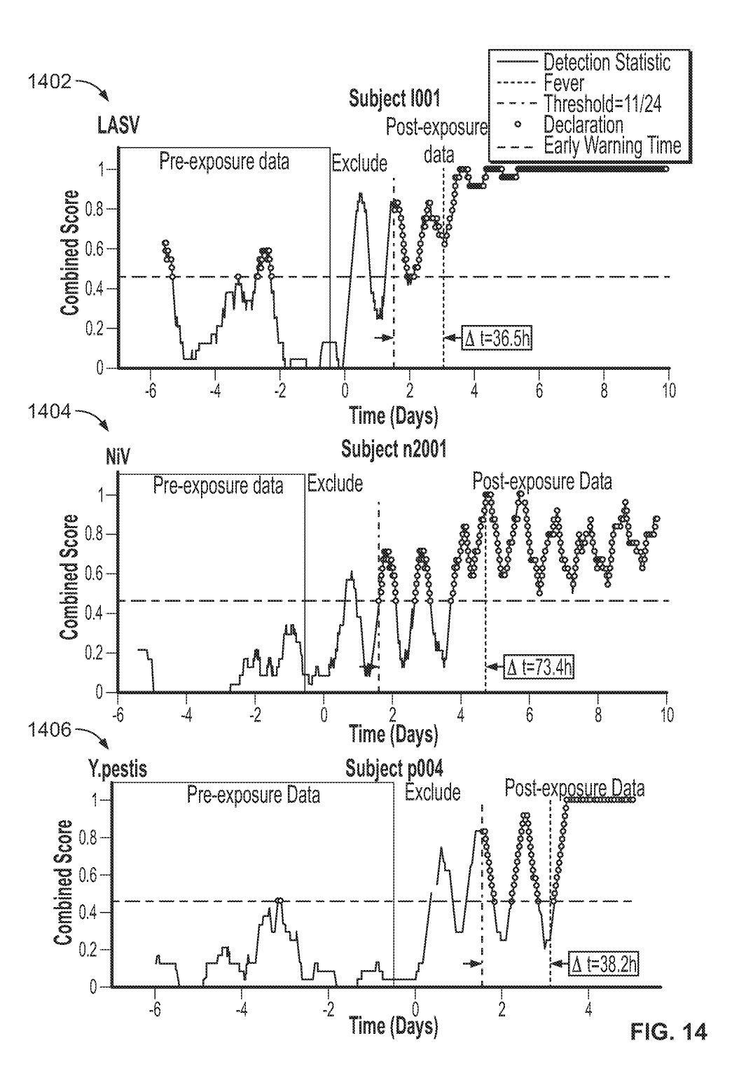

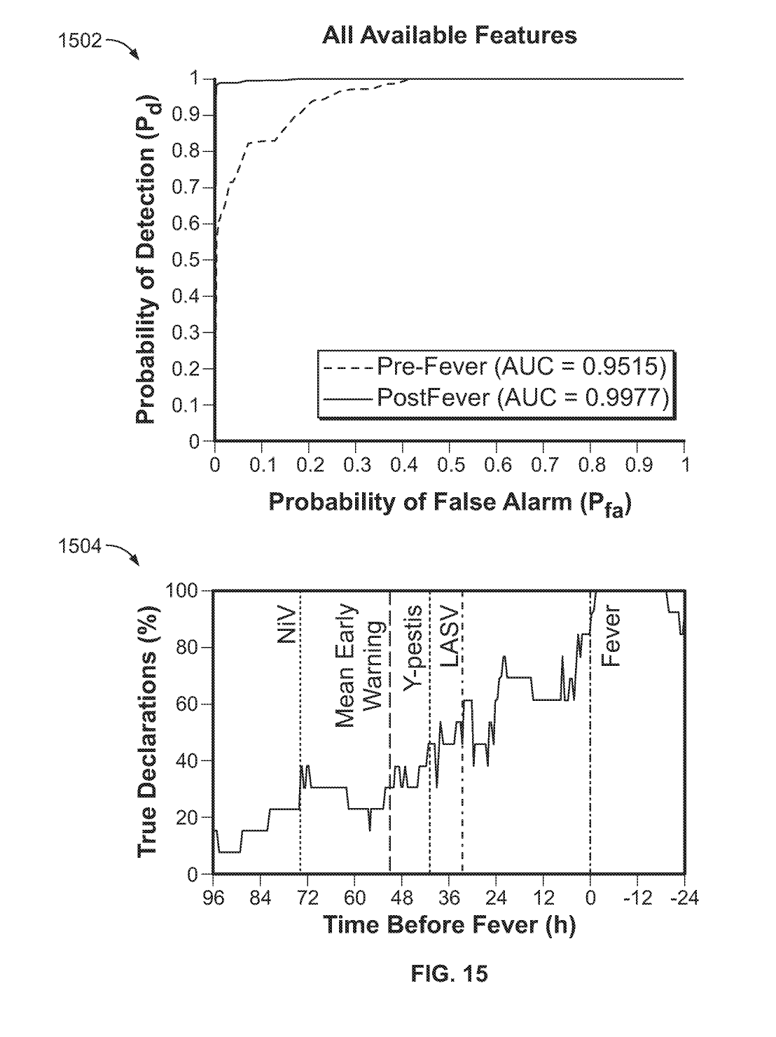

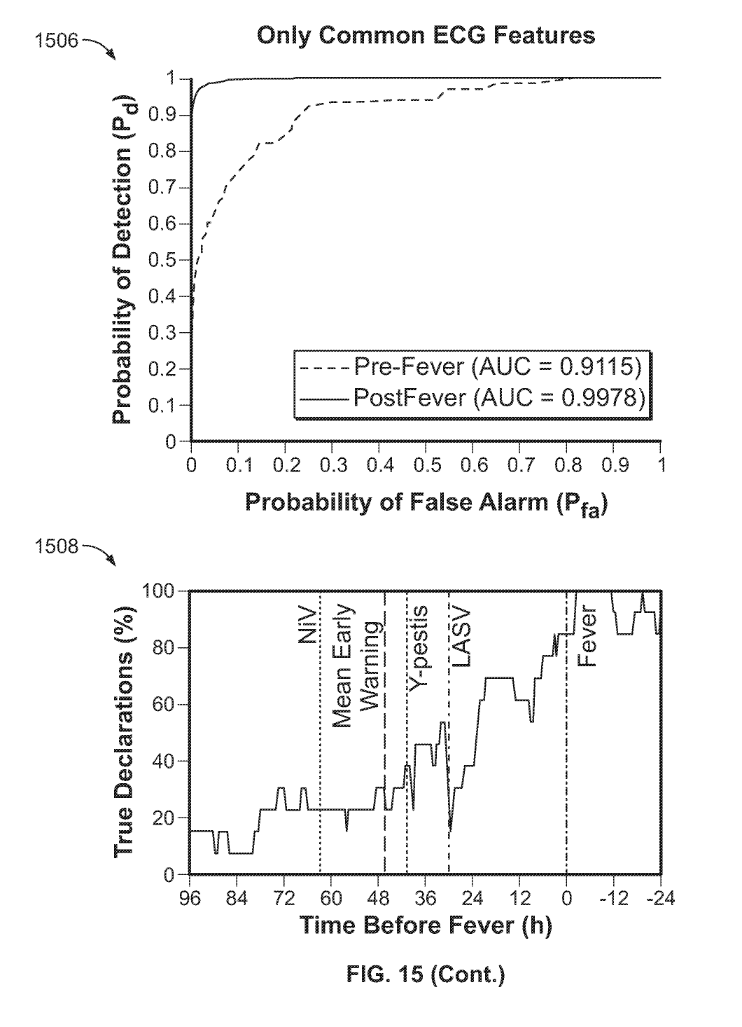

At step 720, a declaration of exposure is provided when the aggregate classifier score for the k-th time interval exceeds a second threshold. In particular, the second threshold may be selected to be a specific fraction m/n. The value for m may be selected in a manner that provides an optimal value, as is described below in relation to Experiment 1 and FIG. 16. When the second threshold is exceeded, a declaration indication is provided at step 720 to indicate that the patient has been exposed to the agent. FIGS. 13 and 14 show exemplary detections (plots 1302, 1402, 1404, and 1406), and FIGS. 13 and 15 show exemplary declarations (plots 1306, 1504, and 1508).

FIG. 8 is a flow diagram depicting a method 800 for predicting whether a patient has been exposed to an agent, according to an illustrative implementation of the disclosure. The method 800 includes the steps of receiving, by at least one processor, physiological data regarding the patient that was recorded during the respective time interval (step 802), extracting one or more features from the physiological data, wherein each feature is representative of the physiological data during the respective time interval (step 804), identifying a plurality of classifiers, each trained using training data for a respective physiological state (step 806), applying each respective classifier to the one or more features to obtain a classifier output that represents a likelihood that the patient has been exposed to the agent (step 808), applying a respective first threshold to each respective classifier's output to determine a patient state classification (step 810), aggregating the patient state classifications across a number of time intervals to obtain an aggregate patient state classification for each respective classifier (step 812), combining the aggregate patient state classifications across the plurality of classifiers to obtain a combined classification (step 814), and providing an indication that the patient has been exposed to the agent when the combined classification exceeds a second threshold (step 816). The steps 802-816 may be repeated for additional respective time intervals.

At step 802, the patient's physiological data that was recorded during a respective time interval is received. As described herein, the physiological data may include pulse data, ECG data, pulmonary data, blood pressure data, temperature data, and any other type of data that is physiologically recorded from the patient. In an example, the physiological data solely includes data that is capable of being recorded from one or more non-invasive wearable devices on the patient. In particular, the physiological data may solely include an electrocardiogram signal, a temperature signal, or both. As used herein, a non-invasive wearable device includes devices that are not implanted into the body and may include devices that are worn or attached external to the body and are capable of sensing or recording physiological measurements from the body. In an example, a non-invasive wearable device may be configured to take marginally invasive measurements, such as oral measurements, buccal measurements, sublingual measurements, rectal measurements, or a combination thereof.

At step 804, one or more features are extracted from the physiological data, wherein each feature is representative of the physiological data during the respective time interval. Specifically, a feature may include one or more summary statistics for the physiological waveforms that are recorded from the patient. In some embodiments, the physiological data is first pre-processed to transform the raw waveforms into values that may be compared across different time intervals. For example, as is described herein, the pre-processing may include standardization techniques to remove short term fluctuations and/or diurnal patterns in the data. This processing may be performed in order to enable the extracted features to be compared across different time intervals. For each time interval, a mean value, a standard deviation, and quartiles of the data values, which may be percent differences, are calculated. These statistics may be used as the features that characterize the physiological data and are representative of the data during the specific time interval during which the corresponding physiological data was recorded. Moreover, the features may also be indicative of the physiological data that was recorded during previous time intervals. In one example, the one or more features include solely heart rate and temperature.

At step 806, a plurality of classifiers is identified. Each classifier is trained using training data for a respective physiological state. In an example, the plurality of classifiers includes two classifiers, where a first classifier is trained using pre-fever training data, and a second classifier is trained using post-fever training data. The pre-fever training data may include all data that is collected before an onset of a symptom, or any subset of such data. For example, the pre-fever training data may include solely pre-exposure data, post-exposure and pre-fever data, or a combination of both. Similarly, the post-fever training data may include all data that is collected after the onset of the symptom, or any subset of such data. For example, the post-fever training data may include only data that is recorded during a specific time interval after onset of the symptom, such as 0-12 hours after fever occurs. In general, the specific time interval after onset of the symptom may include 0-24 hours, 12-24 hours, 12-36 hours, or any other suitable time interval that starts and ends after the symptom occurs.

In an example, the plurality of classifiers further includes a third classifier that is trained using training data following the pre-fever training data and preceding the post-fever training data. This training data used for the third classifier includes physiological data recorded from the patient during a transition period that may begin before the onset of the symptom and ends after the onset of the symptom. The duration of the transition period may be any suitable time interval, such as 12 hours, 24 hours, 36 hours, 48 hours, or any other suitable number of hours. In general, the pre-fever training data, the post-fever training data, and the transition period training data may include data that is recorded over time intervals that have the same or different durations. For instance, the same time interval may be used, such as a single day. In this case, the pre-fever training data is recorded over a 24-hour period before the onset of the symptom, the post-fever training data is recorded over a 24-hour period after the onset of the symptom, and the transition period training data is recoded over a 24-hour period that includes the onset of the symptom.

In an example, the agent is a first agent, and the training data includes data that is recoded from subjects that were exposed to a second agent that is different from the first agent. In this case, there may be a large amount of training data available for the second agent, but relatively less training data available for the first agent. If both agents cause similar biological effects, then classifiers that are trained on exposure to one agent may be used to predict whether exposure to the other agent has occurred.

In an example, the patient is a human, and the training data includes data from non-human animal subjects. In this case, there may be a large amount of training data available that has been recorded from non-human animal subjects, but relatively less training data available that has been recorded from humans. If the non-human animal subjects that provide the training data have similar biological mechanisms as humans (such as primates, for example), then the same features may be used to predict exposure to an agent.

At step 808, each respective classifier is applied to the one or more features to obtain a classifier output that represents a likelihood that the patient has been exposed to the agent. The likelihood may be representative of a probability ranging from 0 to 1. The probability represents a prediction that the recorded physiological data from the patient resembles the training data for a particular physiological state.

At step 810, a respective first threshold is applied to each respective classifier's output to determine a patient state classification. As is described in detail in relation to Experiment 1, the respective first threshold may be determined based on a desired probability of false alarm for each respective classifier.

At step 812, the patient state classifications are aggregated across a number of time intervals to obtain an aggregate patient state classification for each respective classifier. In an example, the patient state classification is a binary value indicative of a prediction by the respective classifier of whether the patient is exposed or not exposed, and the aggregating in includes summing across the binary values. In some embodiments, the aggregating further includes normalizing the summed binary values by the number of time intervals to obtain an averaged score for each respective classifier.

At step 814, the aggregate patient state classifications are combined across the plurality of classifiers to obtain a combined classification, which may be referred to herein as an aggregate classifier score. In some embodiments, the combining includes determining a maximum averaged score across the plurality of classifiers. In general, another statistic other than the maximum averaged score may be used, such as a mean or a rolling average. The combined classification may correspond to some metric that includes an integration of a function over time, in which recent values may be more heavily weighted than older values.

At step 816, an indication that the patient has been exposed to the agent is provided when the combined classification exceeds a second threshold. As is described herein, the second threshold may be determined based on a performance metric of the system that is related to a probability of false alarm, a probability of detection, or early warning purity. In an example, the second threshold may be determined based on a ratio m/n, where n is the number of time intervals and m is an integer greater than 0 and less than or equal to n. This is described in detail in relation to Experiment 1 below.

Experiment 1—Introduction

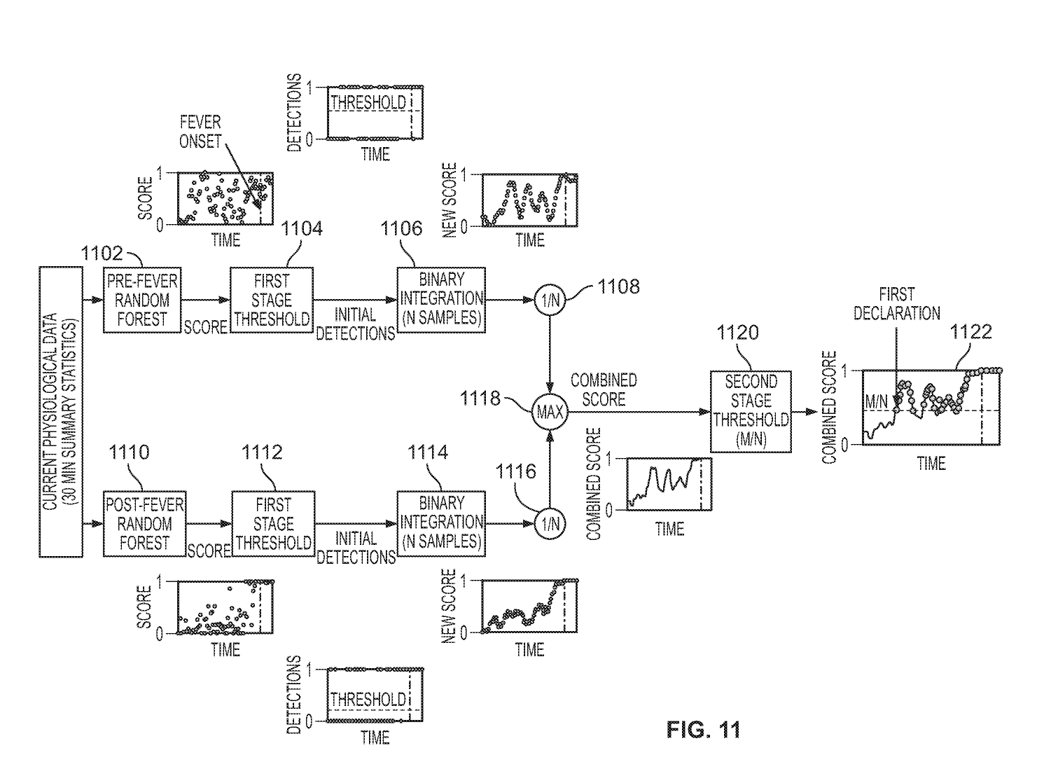

In an exemplary implementation, an experiment is performed involving non-human primate (NHP) subjects. High-resolution (both fast sampling rates and finely quantized amplitudes) physiological data is collected from non-human primates (NHPs) exposed via intramuscular (IM), aerosol, or intratracheal routes to one of several viral hemorrhagic fevers (Ebola virus [EBOV], Marburg virus [MARV], Lassa virus [LASV]), Nipah virus (NiV), or one bacterial pathogen (Y. pestis) to build a high sensitivity, low etiological specificity (i.e., not informative of particular pathogens) processing and detection technique. Physiological data is standardized to remove diurnal rhythms, aggregated to reduce short-term fluctuations, and then provided to a supervised binary classification (exposed and unexposed classes) machine learning technique as illustrated in FIG. 10.

FIG. 10 is a flow diagram of a process for performing a machine learning technique, according to an illustrative embodiment. Specifically, FIG. 10 includes receiving training data (step 1002), which includes data recorded from subjects having a known exposure state (e.g., exposed or not exposed) and receiving test data (step 1006), which includes data recorded from a subject whose exposure state is unknown. Machine learning models are trained, such as random forest classifiers at step 1004. Several methods are tested and compared. In this experiment, random forests exhibit the best positive predictive value and were chosen for the rest of the analysis. Random forests may also be chosen for their robustness to many correlated features while minimizing over-fitting. Random forests are trained (or grown) at two post-exposure stages, thus allowing for adaptation to physiological changes between incubation and prodromal phases. Specifically, one random forest is trained using post-exposure but pre-fever physiological data, and another random forest is trained using post-exposure, post-fever data. Both random forest training sets include pre-exposure data to build the unexposed class. For evaluation, subject data is separated into various training and testing sets, and every testing subject's data is provided to the random forest model for an exposure prediction every 30 minutes. After the machine learning models are trained at step 1004, the declaration logic applies the models and error reduction techniques at step 1008, and finally a prediction is provided regarding whether the testing subject has been exposed or not exposed at step 1010.

FIG. 11 is a block diagram of an example binary integration and thresholding approach to reduce false alarms, according to an illustrative embodiment. Specifically, FIG. 11 includes receiving current physiological data in 30 minute intervals. The pre-fever random forest classifier 1102 is applied to the physiological data to provide a score to the first stage threshold 1104, which provides a 0 if the score is below a threshold and a 1 if the score is above the threshold. Similarly, the post-fever random forest classifier 1110 is applied to the physiological data to provide a score to the first stage threshold 1112, which provides a 0 if the score is below a threshold and a 1 if the score is above the threshold. In general, the value of the threshold applied at 1104 and 1112 may be the same or different. Determining an appropriate value for the threshold applied at 1104 and 1112 may include using a constant false alarm thresholding approach, which is described in detail below. After the scores are thresholded at 1104 and 1112, the resulting binary values are integrated at 1106 and 1114 and normalized at 1108 and 1116. The maximum between the two integration results is determined at 1118, and the result is provided to a second stage threshold 1120, which applies a final threshold m/n (described in detail below) to determine whether a declaration of exposure is provided at 1122.

After using binary integration and a constant false alarm thresholding approach to further reduce false alarms, mean exposure declaration times are found to range from 32.6±40.5 h (for LASV) to 74±37 h (for NiV) before the onset of fever (defined as 1.5° C. above a diurnal baseline sustained for two hours). Once the random forests are trained, all physiological data is given to both pre- and post-fever models, without regard to exposure or fever status. In other words, the approach described herein does not require information on exposure or fever times for successful classification and detection. This approach allows for both flexible, multi-modal input features (customizable to the available sensing hardware) and tunable false alarm rates, which offers a unique ability to adjust system performance per user needs. Additionally, the present disclosure leverages supervised classification to learn subtle physiological changes, and continuously monitors for signs of pathogen exposure rather than relying on a single time ‘snapshot’ of subject data.

Experiment 1—Methods

The Marburg Angola isolate used is United States Army Medical Research Institute of Infectious Diseases (USAMRIID) challenge stock “R17214” (Marburg virus/H.sapiens-tc/ANG/2005/Angola-1379c). This is used for both aerosol (rhesus macaques) and IM (cynomolgus macaques) studies. Cynomolgus macaques are exposed to Ebola virus/H.sapiens-tc/COD/1995/Kikwit-9510621 at a target dose of 100 pfu (7U EBOV; USAMRIID challenge stock “R4415”; GenBank # KT762962). African green monkeys are exposed to the Malaysian Strain of Nipah virus (isolated from a patient from the 1998-1999 outbreak in Malaysia, provided to USAMRIID by the Centers for Disease Control and Prevention). Cynomolgus macaques are exposed to the Josiah strain of the Lassa virus challenge stock “AIMS 17294” (GenBank #s JN650517.1, JN650518.1).

Description of Animal Studies. Physiological data is provided in NSS format (Notocord Systems, Croissy-sur-Seine, France) from adult (non-juvenile) non-human primate natural history studies conducted at the USAMRIID Research is conducted under an IACUC approved protocol in compliance with the Animal Welfare Act, PHS Policy, and other Federal statutes and regulations relating to animals and experiments involving animals. A minimum number of subjects in MARV and EBOV studies is chosen using a Fisher exact test, with 100% lethality as the pre-specified effect. Subjects are randomized for inclusion and pathogen exposure order by age, weight, and gender. No sham control subjects are included in the study design, and pre-exposure data is used to build the “un-exposed” class. In each study, remote telemetry devices (Konigsberg Instruments, Inc., T27F or T37F, or Data Sciences International Inc. L11: see details in Table 1 below) are implanted 3 to 5 months before exposure, and, if used, a central venous catheter is implanted 2 to 4 weeks before. NHPs are transferred into BSL4 containment 5 to 7 days before viral exposure, and baseline pre-exposed data is collected for 4 to 6 days before exposure. Subjects are exposed under sedation via aerosol, intramuscular injection, or intratracheal exposure depending on the study. The exposure time (t=0) used in the model is based upon the time of intramuscular injection or intratracheal exposure, or when a subject is returned to the cage following aerosol exposure (˜20 min). All subjects are monitored until death or the completion of the study. Since these natural history studies involve no diagnostic tests or therapeutic interventions, and all subjects are administered infectious doses, there is no need for investigator blinding during the data collection phase. Investigators are blinded to the study design until after animal data collection. The telemetry devices measure several raw physiological signals, which are translated to blood pressure (sampling frequency fₛ=250 Hz), ECG (fₛ=500 Hz), temperature (fₛ=50 Hz), and pulmonary (fₛ=50 Hz) features using Notocord software. Six separate exposure studies are conducted. The studies use all subjects' post-exposure data that had sufficient fidelity (i.e., no data loss from equipment failure), which developed fever two days or less before the studies' mean (i.e., no possible co-morbid infections), and did not receive a post-exposure therapeutic. These criteria lead to 13 excluded animals, 2 from each the NiV and MARV IM studies, and 9 from the EBOV study (including 7 which received therapy). Some of the excluded EBOV and NiV subject's pre-challenge data are used in the independent dataset validations to estimate thresholds and reduce the false alarm rate.

TABLE 1

| Pathogen | Exposure | Subjects | | Monitoring | Target |
|---|---|---|---|---|---|
| (reference) | method | (m/f) | Species | system | dose |
| EBOV | Aerosol | 6 (3/3) | Cynomolgus | 3 subjects | 100 pfu |
| | | | | with | |
| | | | | ITS T37F | |
| | | | | 3 subjects | |
| | | | | with | |
| | | | | DSI L11 | |
| MARV | Aerosol | 5 (3/2) | Rhesus | ITS T27F | 1000 pfu |
| (75) | | | | | |
| MARV | IM | 9 (7/2) | Cynomolgus | ITS T27F | 1000 pfu |
| NiV (74) | IT | 5 (5/0) | African | ITS T27F | 20000 pfu |
| | | | green | | |
| | | | monkey | | |
| LASV | Aerosol | 4 (4/0) | Cynomolgus | ITS T27F | 1000 pfu |
| (27) | | | | | |
| Y. pestis | Aerosol | 4 (4/0) | African | ITS T27F | 100 LD₅₀ |
| | | | green | | |
| | | | monkey | | |

Physiological Data Processing.

All data processing and modeling is performed in Matlab (MathWorks, Natick Mass.). Physiological data is time dependent (that is, sequential time-series data) and is subject to short-term fluctuations and diurnal or circadian rhythms. Random forest classifiers, however, assume that the statistics of the data are independent of time and subject. In other words, the physiological data may be pre-processed to remove this time dependence, to allow for useful comparison of the features of the physiological data across different time intervals. To reduce diurnal and subject-to-subject dependencies from the data, each subject is pre-processed individually. The first processing step is to remove artifacts from motion, poor sensor placement or intermittent transmission drop outs by dividing the data into a series of k-minute intervals and omitting the top and bottom 2% quantiles for each interval. Next, baseline diurnal statistics are estimated for the i^{th} time-of-day interval during the pre-exposure period (i.e., data from several pre-exposure days, all corresponding to the same time of day, such as the thirty minute interval from 12:00 PM to 12:30 PM) by computing mean, μᵢ, and standard deviation, σᵢ. The data for the i^{th} time-of-day interval is standardized by subtracting the mean and dividing by the standard deviation from each data sample xᵢ(j) in the i^{th} interval, (xᵢ(j)−μᵢ)σᵢ. For a sufficiently short time interval of k-minutes, the data statistics are assumed to be approximately constant, therefore standardization mitigates diurnal time dependence from the signals. Then, three summary statistics are calculated for an l-minute block: mean and 25% and 75% quantiles. These time-independent summary statistics are the features for the random forest algorithm. The influence of values for k and l on successful classification are investigated. While k and l do not need to be identical, k=l=30 minutes is chosen as a trade off between computational requirements and low random forest out-of-bag-errors. For example, k=l=30 min for two days of 4 raw physiological signals yields 96 time points with 12 data features. Data samples that correspond to measurements before pathogen challenge are labeled “0” to denote the pre-exposed class and those after challenge are labeled “1” to denote the post-exposure class.

Random Forest Ensemble.

The model consists of two random forests. One random forest is grown using post-exposure training data prior to fever onset (labeled class “1”) and an equal number of randomly chosen negative data samples from the pre-exposure period (class “0”). The second random forest is trained similarly, but class “1” data corresponds to post-exposure training data after fever onset. Test data is held out until the final evaluation step. Each random forest contains 15 classification decision trees grown on random subsets of data and features. 15 trees are chosen as a trade off between model over-fitting and successful classification, as indicated by random forest out-of-bag-errors. The trees cast their “votes” for class “0” or “1,” and the forest returns a score equal to the proportion of trees that voted for the exposure (“1”) class. This process helps prevent overfitting, which is a common concern for single decision trees. Random forests are useful for calculating feature importance metrics, and these metrics are used to find the most predictive features for difficult-to-classify pre-fever days. Initially all features are considered for training the random forest models, but once a subset of most predictive features is determined within a cross-validation training set, the random forests are regrown (on the original training dataset) using only the top 10 features to produce the final models upon which the corresponding testing set performance results are based. A rank order list of top 10 features from each study is provided in Tables 4 and 5 below, with legends provided in Tables 2 and 3 below.

TABLE 2

| Feature | |
|---|---|
| Name Prefix | Description |
| APP | Area of positive thoracic pressure during each respiratory |
| | cycle, corresponding to inhalation |
| ANP | Area of negative thoracic pressure during each |
| | respiratory cycle, corresponding to exhalation |
| AOPAMean | Approximated mean arterial pressure between two |
| | successive diastoles (=⅓ * P_{systolic} + ⅔ * P_{diastolic}) |
| AOPDiastolic | Aortic pressure during diastole |
| AOPSystolic | Aortic pressure during systole |
| Bazett | QT interval corrected per the Bazett method (54) |
| Friderica | QT interval corrected per the Friderica method (53) |
| HR | Heart rate computed between two successive diastoles |
| | from the ECG waveform (inverse of RR) |
| LVPDiastolic | Left ventricular pressure during diastole |
| LVPMean | Arithmetic mean of the left ventricular pressure between |
| | two successive diastoles |
| LVPRate | Heart rate computed between successive local maxima in |
| | left ventricular pressure waveform |
| LVPSystolic | Left ventricular pressure during systole |
| PR | Time interval from P and R points on ECG waveform |
| QRS | Time interval from Q and S points on ECG waveform |
| QT | Time interval from Q and T points on ECG waveform |
| RespMean | Mean respiratory rate calculated over a non-overlapping |
| | 200 s time window |
| RespRate | Instantaneous respiratory rate, computed between two |
| | successive inhalations |
| RR | Time interval from adjacent R points on ECG waveform |
| Temp | Core temperature |

TABLE 3

| Feature Name Suffix | Description |
|---|---|
| Mean | Mean |
| Q25 | 25^{th} quartile |
| Q75 | 75^{th} quartile |

TABLE 4

| | Aggregated 3-fold cross-validation | | |
|---|---|---|---|
| | Partition 1 | Partition 2 | Partition 3 |
| Pre- | AOPDiastolic_Q25 | PR_Mean | Temp_Mean |
| fever | AOPSystolic_Q75 | RR_Q75 | RR_Mean |
| | Temp_Mean | QT_Q25 | PR_Mean |
| | PR_Mean | Bazett_Q25 | QT_Q75 |
| | Bazett_Mean | Temp_Mean | AOPSystolic_Q75 |
| | RR_Mean | QRS_Mean | PR_Q75 |
| | Temp_Q25 | QRS_Q25 | QT_Mean |
| | Bazett_Q25 | QRS_Q75 | HR_Mean |
| | Fridericia_Mean | PR_Q25 | Bazett_Mean |
| | AOPDiastolic_Mean | Temp_Q25 | RespMean_Mean |
| Post- | Temp_Mean | Temp_Mean | Temp_Mean |
| fever | PR_Mean | AOPSystolic_Mean | Temp_Q75 |
| | Temp_Q25 | Temp_Q75 | AOPDiastolic Mean |
| | AOPDiastolic_Q75 | AOPSystolic_Q75 | AOPSystolic_Q75 |
| | Temp_Q75 | Temp_Q25 | RR_Q75 |
| | RespMean_Mean | AOPSystolic_Q25 | Temp_Q25 |
| | RR_Mean | AOPAMean_Mean | AOPDiastolic_Q75 |
| | AOPDiastolic_Mean | QT_Q75 | AOPDiastolic_Q25 |
| | RespMean_Q75 | HR_Q25 | QT_Q75 |
| | RR_Q75 | RR_Q75 | HR_Mean |

TABLE 5
Independent Dataset Validations

| Pre-Fever | Post-Fever |
|---|---|
| QRS_Mean | Temp_Mean |
| Temp_Mean | RR_Q75 |
| RR_Q75 | AOPAMean_Mean |
| AOPDiastolic_Q25 | QRS_Mean |
| AOPAMean_Q25 | AOPDiastolic_Mean |
| Bazett_Mean | Bazett_Q25 |
| QT_Q25 | PR_Mean |
| PR_Q25 | AOPSystolic_Mean |
| AOPDiastolic_Mean | Bazett_Mean |
| QT_Mean | AOPDiastolic_Q25 |

Detection Logic.

Declarations of exposure are made using a two-stage detection process, as described in relation to FIG. 11. In stage one of the detection process, random forest model prediction scores (between 0 and 1 for every l=30 minute interval) are thresholded (i.e., a value of 1 is returned if the random forest model score is greater than or equal to a false alarm rate determined threshold, discussed below) to form a series of initial detections for the model every l=30 minutes. Threshold levels for both pre- and post-fever random forests are estimated by analyzing false alarm rates (Type I errors) of the initial detections versus threshold levels (swept from 0 to 1). The probability of false alarm (or P_{fa}) is defined as:

$${P}_{fa} = \frac{\# False Positives}{\# True Negatives + \# False Positives}$$

To enforce a desired significance level (such as P_{fa}=0.01, for example), a threshold is estimated as a value needed to achieve a target P_{fa} using a 3-fold approach similar to that used in random forest model training. For the case of validating performance on an independent test set (NiV, LASV, and Y. pestis), the test set subjects are randomly assigned into 3 partitions for the purposes of threshold estimation. This approach maintains separation between the partition-under-test and the remaining two partitions used for threshold estimation, while providing a sufficient number of samples to estimate low rates of false alarms. Detections from the unexposed class of all but the partition-under-test are used to select the smallest first-stage thresholds (for pre- and post-fever as seen in FIG. 11) that support the desired P_{fa}. This approach is repeated for each partition, resulting in independent estimates of the threshold pair (pre- and post-fever) for each partition. While a significance level of P_{fa}=0.01 is targeted, the overall system P_{fa} may be higher or lower due to strict separation between the subjects-under-test and the subjects used to estimate the threshold.

These initial detections from each random forest model are subjected to a second-stage detection test to further reduce the false alarm rate. During the second stage, binary integration is performed over a sliding window of the past n initial detections. The accumulated detections are divided by n, giving a mean score for the pre- and post-fever random forest models. Next, scores are combined by taking the maximum of the pre- or post-fever values to create a single time series. At each 30 minute time interval, this combined score is compared to a final declaration threshold of m/n, where m≦n (in this example, n=24 for a system latency of no more than 12 hours and m=11 which approximates the optimum binary integration threshold for a steady signal in noise; performance is relatively insensitive to small deviations in m or n). In general, m and n can take on any integer values. A ‘declaration’ is made that the subject is in the exposed class when the combined score is greater than or equal to m/n. Alternatively, if the threshold is not met, the subject is assigned to the ‘not exposed’ class for that time epoch. In general, n samples are required before a declaration can be made, so following the start of data collection or the end of an exclusion period (the 24 hour period following the challenge), no declarations are reported in the first k*n minutes (for n=24 and k=30 min, this accumulation period effectively extends the exclusion period to 36 hours post-exposure).

Model Performance Evaluation: Three-fold Cross-Validation and Independent Dataset Testing.

Model performance may be evaluated by strictly separating subjects into testing and training sets. To characterize the performance, two modes of evaluation are conducted: 1) a three-fold cross-validation, where a collection of exposure studies is used to develop and test the algorithms (data includes EBOV aerosol, MARV aerosol, MARV IM, and thus can vary in subject species, virus, and exposure route conditions), and 2) an independent validation where models trained on the initial set of exposure studies (used in (1) above) are applied to an entirely new dataset with pathogens and experimental conditions not seen in the models' training or tuning.

In the three-fold cross-validation mode of evaluation, subjects from the aggregated collection are randomly assigned into three partitions (each partition included animals from each of the 3 constituent exposure studies), which has been shown to perform better than leave-one-out validations for smaller datasets. In turn, subjects from one partition are used to train the random forest models, the second partition was used as an independent cross-validation set to evaluate effects of tuning the model and algorithm parameters, and the third partition was used to evaluate final model performance. Model building and performance evaluation is repeated three times such that each partition is evaluated in each role. For mode (2), independent dataset testing is performed by treating all subjects from the three studies used in the initial set (EBOV, MARV IM and MARV aerosol) as a single training set to build and the random forest models and select the most important features. The resulting random forest models are then applied to previously unseen subjects from the LASV, NiV, and Y. pestis studies for the final performance analysis.

To evaluate system-level performance, probability of correct declaration P_{d} is defined as:

$${P}_{d} = \frac{\# True Positives}{\# True Positives + \# False Negatives}$$

and P_{fa} as above, where the True Positives, False Positives, True Negatives and False Negatives are evaluated on the final declaration outputs of the block diagram shown in FIG. 11. When reporting P_{d} and P_{fa} for a study and exposure condition, the 95% confidence interval is reported and is based on normal distributions since the number of trials per study is large (>500 declaration points per class). Although some correlation is likely within a binary integration window of k*n minutes, independence may be assumed for trials separated by at least k*n minutes. Receiver operating characteristic (ROC) curves are generated to measure system performance by calculating P_{d} vs P_{fa} at a series of threshold values (sweeping the first-stage detection threshold but holding the second-stage m/n threshold constant) and quantifying the system performance with the ROC area under the curve (AUC), where an AUC=1.0 indicates perfect performance and AUC=0.5 indicates that the model is no better than a coin toss. Sensitivity (P_{d}) is expected to be highest after febrile symptoms are apparent. To distinguish the sensitivity of the system during the pre- and post-fever epochs, P_{d} is calculated independently for subsets of positive data that occur before and after the onset of fever. The result is two ROC curves and corresponding AUCs: one evaluated on positive data restricted to pre-fever time samples and the another restricted to post-fever time samples. The negative data and two-stage detection process are identical for both ROC curves.

In a clinical early warning system, it may be desirable to calculate P_{d} and P_{fa} on a per-device, per-subject, or per-day basis. However, for this proof-of-concept study, the limited pool of subjects available (N=33 total) involved calculating P_{d} and P_{fa} across all 30-minute test points that are not in the exclusion window (12 hours before and 24 hours after exposure). This approach includes false negatives that may occur after an initial early warning declaration is made, and thus provides a conservative estimate of the device sensitivity which may further increase with larger sample sizes and more refined processing techniques.

Another important measure of system performance is the mean early warning time. The early warning time for an individual subject is defined as the time of the first true declaration (excluding data from the 24 h interval immediately following the challenge) minus the time of fever onset (defined as 1.5° C. above a diurnal baseline sustained for two hours). Early warning times vary across subjects in a study, so the mean value is calculated across all subjects to characterize the early warning time afforded by the system. Since the number of trials (equal to the number of subjects) for this performance metric is relatively small, the mean early warning time is bounded with a 95% confidence interval based on a t-distribution. Mean Δt is an unstable performance metric when evaluating small subsets of the data, such as on a per-pathogen level.

Model tuning, including feature selection and other classifier and detection parameters, may also be performed using an independent cross-validation testing set. For example, FIG. 16 includes performance evaluation results across different detection logic parameters m and n for a target system where P_{fa}=0.01. Specifically, a theoretical optimal value of m for a given n and P_{fa} is indicated by the dashed lines, and an operating point of Experiment 1 is indicated by an asterisk. The four plots in FIG. 16 are related to an early warning time (plot 1602), pre-fever probability of detection P_{d} (plot 1604), false positives (plot 1606), and pre-fever AUC (plot 1608). The plot 1602 shows that small values of n promote earlier warning times by limiting the evaluation interval for a declaration of exposure. The plot 1604 shows that the theoretical optimal value for m for a given n and P_{fa} aligns with a relatively flat region of high P_{d}. The plot 1606 shows that the actual system P_{fa} is a few percent higher than the target system P_{fa} of 0.01, but is relatively insensitive to the choice of m and n (except for very small ratios of m/n). The plot 1608 shows that the overall detection performance (as measured by an ROCAUC metric) improves with larger values of n. The various plots in FIG. 16 illustrate some of the design trade-offs in selecting a short enough evaluation interval to allow for early warning while enforcing a long enough interval to maintain low false positives and high detection sensitivity prior to fever.

Experiment 1—Results

Data Preprocessing and Detection.

High resolution (both temporally and amplitude sensitivity) physiological waveform data are collected during previously conducted natural history studies (detailed in Table 1) at the United States Army Medical Research Institute of Infectious Diseases (USAMRIID) to build a binary classification random forest model for detecting whether an animal had been exposed to a pathogen (either EBOV, MARV, LASV, NiV, or Y. pestis). Supervised machine learning techniques learn data characteristics that belong to pre-determined classes, then place new, unseen data into the appropriate class based on similar characteristics. Pre- and post-exposure are defined as the two classes since “infection” itself is not a discrete event and all exposures in these studies lead to infection and illness.

Several classification methods are tested, including Naïve Bayes, k-Nearest Neighbors, and random forests, and compared each across sensitivity, specificity, and early warning time metrics. While all the tested classifiers have positive predictive values, random forests are chosen for several reasons. Importantly, random forests require no assumptions about the statistical independence of features, which is useful given highly correlated physiological feature sets. They also allow for the calculation of quantitative feature performance. This facilitates post-hoc comparison to the known viral pathology sequence to mechanistically understand why these physiological anomalies are present, and which sensor types provide the most value. Furthermore, the most discriminating features can be selectively chosen to re-grow forests and allow for better algorithm performance with fewer feature inputs, helpful in addressing the dilemma of having many more features than samples or subjects producing them. Next, because each decision tree in a random forest ensemble is grown on a different subsample of training data, random forests avoid over-fitting (which is commonly seen in single decision trees) and reduces variance. Finally, in empirical comparisons of many machine learning methods, random forests consistently rank among the best approaches, and random forests produce the best outputs among the classifiers tested.

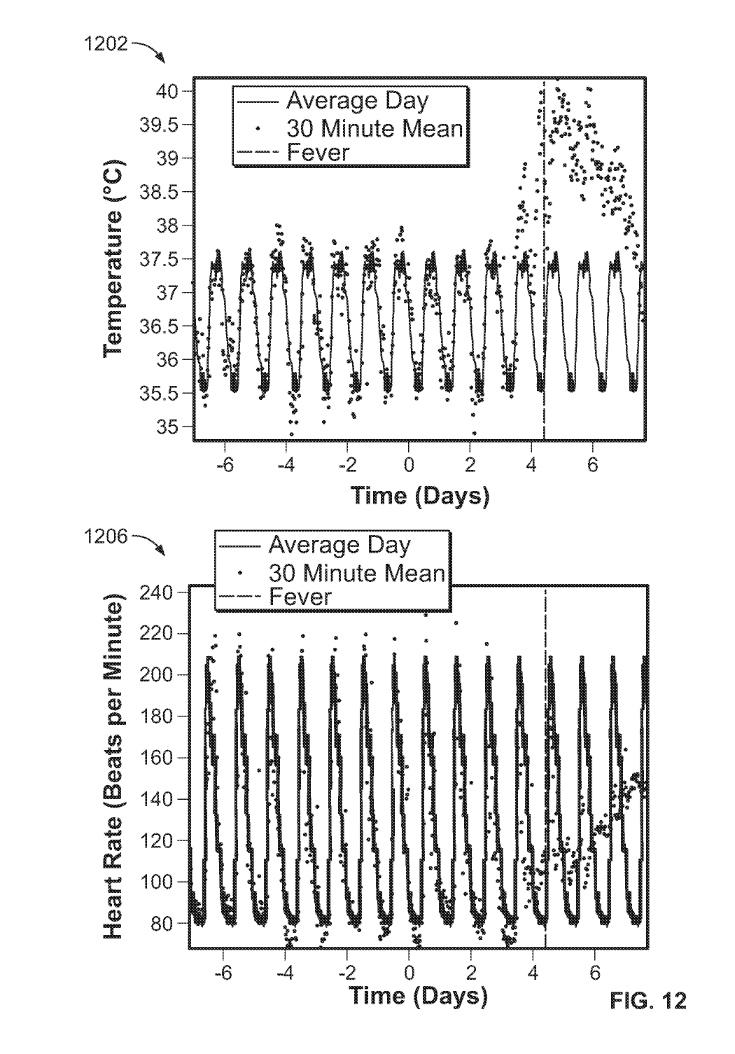

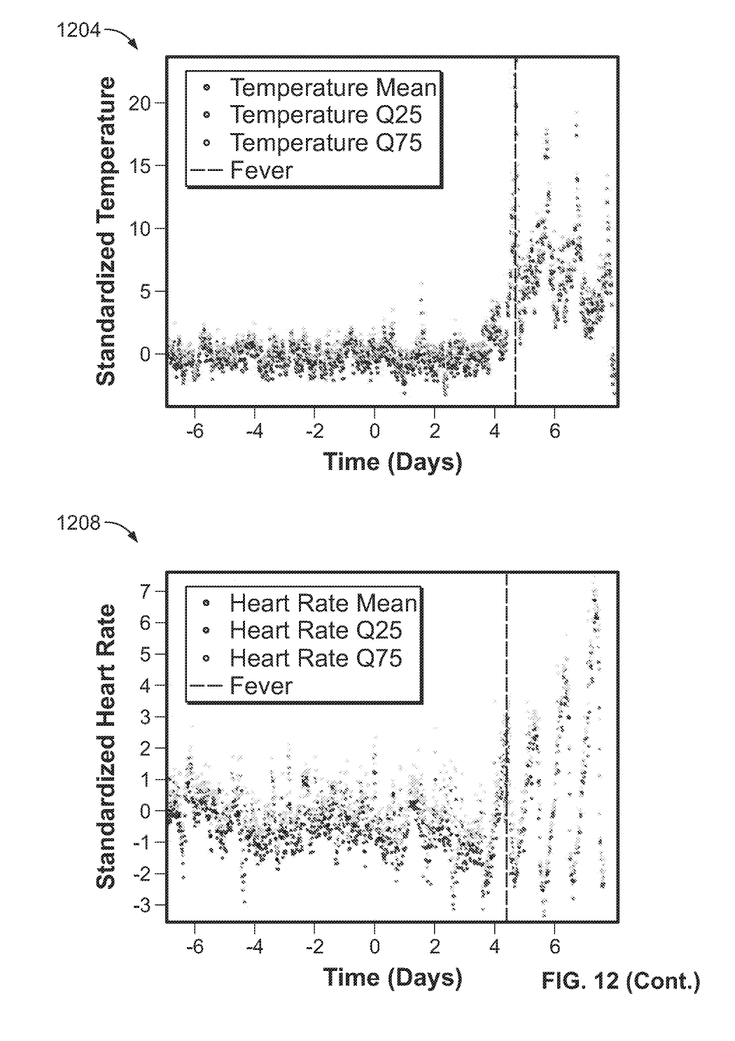

Before classification, several data processing steps are performed to remove time as an implicit feature in the physiological datasets. First, data is standardized and aggregated subject-by-subject to eliminate short-term fluctuations and daily diurnal rhythms. From these standardized datasets, mean and quantiles are calculated for each time window. FIG. 12 includes four exemplary plots of temperatures before standardization (plot 1202) and after standardization (plot 1204) and heart rate before standardization (plot 1206) and after standardization (plot 1208). The temperature and heart rate time courses are plotted every 30 minutes from one subject in the MARV aerosol study. The curves in the plots 1202 and 1206 represent an average diurnal value for this subject before exposure, and the plots 1204 and 1208 show the standardized data after the mean, standard deviation, and quantiles are calculated. The vertical lines in each of the plots 1202, 1204, 1206, and 1208 indicate an onset of fever, defined as 1.5 degrees Celsius above the diurnal baseline sustained for 2 hours. These data are included in the features provided to the machine learning technique.

These statistical measures are the features provided to the machine learning technique (see Table 2 for a complete list of features considered). Windows of length 30 minutes are chosen as a tradeoff between computational requirements and performance (as indicated by random forest out-of-bag errors). For the rest of the analysis, data from 12 hours before and 24 hours after viral or bacterial challenge are excluded from performance metrics due to differences in animal handling and exposure sedation that resulted in significant physiological deviations from baseline data unrelated to pathogen infection.

After data is standardized and aggregated, these features are used to train a random forest classifier. This resultant ensemble is a collection of fifteen binary decision trees which then “vote” on whether given new data belongs in the exposed or unexposed class. In Experiment 1, more than fifteen trees in a random forest do not significantly decrease the out of bag error, which measures classification success. In the final model, two random forests are trained to detect the post-exposure class at distinct time epochs: one model is tuned to detect subtle markers during the incubation phase prior to fever, while the second model is tuned for the early prodromal phase (i.e., onset of overt febrile symptoms) where temperature-related features emerge as powerful discriminants. The training data for the pre-exposure class for both models is a subset of baseline data prior to challenge and the quantity of training data has been balanced for the negative (pre-exposure) and positive (post-exposure) classes to avoid biasing one class over the other. To select the ideal features to put in these final forests, the feature importance metrics are inspected. These metrics are given by random forests built consecutively on a reducing feature set. In this way, the top ten features are selected, ten being the selected by results from a cross-validation set, and the final models are built with these features. The output of these random forest ensembles, however, is prone to false alarms, and a two-stage detection logic process is employed to reduce false positives to a pre-determined target level (such as P_{fa}=0.01, for example). Final declarations of “exposed” or “unexposed” are the output of this two-stage process.

Evaluation: Three-Fold Cross-Validation.

The machine learning approach described herein is developed and tested with three initial exposure study datasets based on MARV IM, MARV aerosol, and EBOV aerosol exposures. Data from across all three studies are aggregated and used to train and test a random forest model in a three-fold cross-validation scheme, where each partition is composed of randomly-selected subjects from each of the three exposure studies (i.e., the group of subjects in a partition is not based the same as a cohort in an exposure study). In doing so, this explicitly varies 4 experimental variables (species of animal, exposure route, pathogen, and target dose) across the three partitions, which reduces the likelihood of biasing the model for any particular condition.

FIG. 13 depicts performance for one representative subject from the MARV aerosol exposure study (whose early warning time is closest to the studies' mean). Plot 1302 includes a curve for the combined score output by the machine learning technique as a function of time, for a pre-exposure time interval 1308, an excluded time interval 1310, and a post-exposure time interval 1312. The circle overlays during the post-exposure time interval 1312 correspond to declarations made by the detection threshold and binary integration methods described herein. The combined score remains below the detection threshold (dashed horizontal line at value 11/24 in the plot 1302) before virus challenge, rises sharply around exposure (which is excluded) due to anesthesia, then rises again at ˜2 days post-exposure when the first “exposed” declaration is made at 1314, which represents the first true positive declaration. If found before pathogen exposure, a declaration would represent a false alarm. Combined score values below the detection threshold after exposure represent false negatives and the time between the first declaration 1314 and fever 1316 is this subject's early warning time Δt. The plot 1304 depicts the ROC curve, indicating nearly perfect performance after febrile symptoms (curve 1318), and strong positive predictive power (AUCROC=0.9343) before fever (curve 1320). The plot 1306 depicts the sensitivity (as measured by a percentage of true declarations versus time before fever, in hours) of the techniques described herein for all 20 subjects, as well as the mean Δt (vertical dashed lines) for each of the three constituent studies. Half of the subjects are correctly identified as exposed 24-36 hours before fever, regardless of the particular pathogen, exposure route, or target dose.

While Δt is clinically very useful, the mean early warning time for these datasets is an unstable performance metric since small changes in the number of subjects and detection logic thresholds can have large impacts on Δtₘₑₐₙ. In this cross-validation scenario, a system probability of detection is identified as P_{d}=0.80±0.01 (i.e., correctly declaring a subject as being exposed after the pathogen challenge), a pre-fever P_{d} is identified as 0.56±0.02, a system probability of false alarm is identified as P_{fa}=0.013±0.003 (i.e., incorrectly declaring a subject as exposed before the pathogen exposure), and Δtₘₑₐₙ=51.0±11.9 h based on 9931 decision points and N=20. As used herein, “early warning purity” refers to a measure of declaration confidence and is a ratio of false negatives to total detection opportunities that occur between the first true positive declaration and before fever, for each subject.

The performance of the techniques described herein are evaluated for all subjects by characterizing the system P_{d} versus P_{fa}, known as a receiver operating characteristic (ROC) curve (as is depicted in the plot 1304 in FIG. 13). ROC curves describe the sensitivity (P_{d}) and specificity (1−P_{fa}, i.e., not informative of the causative agent) of a test and can be partially summarized by the area under the curve (AUC, where AUC=1.0 refers to a perfectly sensitive and specific detector, and AUC=0.5 indicates a test no better than a coin-flip). For this three-fold cross-validation, AUC=0.9343 for the pre-fever model, and AUC=0.9999 for the post-fever model, indicating strong positive predictive value during the “non-symptomatic” incubation period (where early warning is most meaningful) and nearly perfect performance during the symptomatic, febrile prodrome. The final metric for performance is shown the plot 1306 in FIG. 13, which plots the percentage of subjects correctly declared as “exposed” (true positives) vs. early warning time, and is a measure of algorithm declaration sensitivity as a function of time given a target P_{fa}=0.01. Each individual exposure cohort is shown as a dashed vertical line, which indicates individual differences between pathogens (and exposure study conditions). Within these three studies, the earliest mean warning time for MARV IM exposure is at Δtₘₑₐₙ=69 h, and the two aerosol exposures, EBOV and MARV, have similar mean values at Δtₘₑₐₙ=33 h and Δtₘₑₐₙ respectively.

An additional output of the random forest models is a measure of relative feature importance; that is, which features provide the most accurate separation between exposed and non-exposed classes. The most discriminating features for the pre- and post-fever random forest models are identified from a set comprised of four feature types derived from temperature, ECG, blood pressure, and respiration measurements. Table 4 above includes a complete listing of most discriminating features in each model partition. The random forest model reports features that follow clinical symptomology, namely that core temperature-based features (mean and quantiles of temperature) in the post-fever, prodrome model are the highest ranking in importance. Before fever, however, subtle ECG, blood pressure, and temperature derived features seem to be the highest ranking in feature importance, as has been reported at the earliest stages of sepsis (see Discussion below). Among the hemodynamic features, quantiles of systolic and diastolic aortic pressure are among the most important. Among ECG-derived features, means and quantiles of QT intervals (corrected or not), RR intervals (inverse of instantaneous heart rate), and PR intervals are routinely selected as those with the greatest predictive capability. That both inter- and intra-cardiac cycle features are selected, and that the statistical distributions (rather than just the means) of ECG-based features emphasizes the value of high sampling rate waveform analysis, rather than single time point (such as Korotkoff sound based blood pressure) or averaged (heart rate based on observed beats per unit time) measures. Fortunately, ECG and temperature-based features are among the most consistent predictors throughout the six studies considered (since some studies used different monitoring hardware or software configurations), and allow application of these random forest models beyond the exposure studies used to train them.

Evaluation: Testing on Independent Datasets.

The techniques described herein are further able to handle entirely independent data unavailable during model training and development. Whereas in the three-fold cross-validations above, models are tested on a held-out subset of data from within the same exposure studies, models can be also be trained on exposure study datasets and then be tested against entirely independent datasets. These new datasets are collected during studies using different pathogens, animal species, target doses, and exposure routes, just as above, and are collected in separate experimental protocols by different researchers at different times. To perform this type of validation, the random forest models are trained using all subjects from the MARV IM, MARV aerosol, and EBOV aerosol studies, then are tested against unseen data from LASV aerosol, NiV intratracheal, and Y. pestis aerosol exposures. Across all three pathogens, P_{d}=0.90±0.007 and P_{fa}=0.025±0.004, a pre-fever P_{d}=0 0.55±0.03, and a Δtₘₑₐₙ=51.0±13.9 h. FIG. 14 includes plots for one representative subject for each pathogen. Specifically, the plots in FIG. 14 are similar to the plot 1302 in FIG. 13, but the plots in FIG. 14 are related to the independent dataset validations for LASV (plot 1402), NiV (plot 1404), and Y. pestis (plot 1406). The results in FIG. 14 indicate that models that are trained on one type of dataset may be used to predict exposure in different type of dataset.

FIG. 15 includes ROCs and sensitivity plots for the independent dataset validations, according to an illustrative embodiment. Specifically, FIG. 15 includes two plots related to all available features from the implantable telemetry system (plots 1502 and 1504), and two plots related to only features that are derived from the ECG module that were common among all available studies (plots 1506 and 1508). Even though the classifier was trained only on EBOV and MARV, the techniques described herein provided significant pre-fever positive predictive value, with an AUCROC=0.9515 (plot 1502). The plots 1504 and 1508 each depicts sensitivity vs. time curve for all subjects in the independent datasets, along with mean Δt for each pathogen exposure study. For all available features, the plot 1504 indicates that NiV has the longest tₘₑₐₙ=74 hours (though NiV subjects also have the longest incubation period, ˜5 days, and often these subjects have mediocre early warning purity values). When only common ECG features are considered, the plot 1506 indicates that LASV and Y. pestis exposure studies have Δtₘₑₐₙ=33 hours and Δtₘₑₐₙ=41 hours, respectively (with a mean incubation period ˜3.5 days). In addition to testing against subjects exposed to independent pathogens, the dataset is supplemented with un-exposed, pre-challenge subject data from the EBOV and NiV studies that are otherwise excluded. These data include seven full days of measurements from nine animals prior to pathogen exposure: 7 subjects from the EBOV study (excluded due to therapeutic intervention following exposure) and 2 subjects from the NiV study (which developed fever earlier than our exclusion criteria). Detection results on these sham data result in a consistently low false positive rate of P_{fa}=0.017±0.005.

Using these independent validation sets, the random forest models trained on the original set of EBOV and MARV exposure studies continue to provide clinically useful early warning times with a manageable false alarm rate even against pathogens, exposure routes, or animal species that were unavailable during training. This successful extension of an early warning classifier trained on EBOV and MARV for a hemorrhagic fever virus (LASV), a henipavirus (NiV), and a gram-negative coccobacillus (Y. pestis) suggests insensitivity of the systems and methods of the present disclosure to particular pathogens, and possible generalization for novel or emerging agents for which data has not or can not be collected.

Extending to Non-Invasive Monitoring Platforms.

Physiological data features are collected using surgically implanted monitoring devices. Such data would not be expected from military service members, health care workers responding to an outbreak, hospital patients, or the general public. As an in silico simulation for limiting our dataset to what may be collected using a wearable monitoring device, the considered feature set is reduced to include only ECG-derived features such as RR, QT, QRS, and PR intervals. FIG. 15 compares the performance of the techniques described herein using all available features (plots 1502 and 1504 in FIG. 15) and features derived only from the ECG waveform (plots 1506 and 1508 in FIG. 15). Only modest performance decreases are observed in Δtₘₑₐₙ (46.0±14.1 h), pre-fever P_{d} (0.55±0.03), and system P_{d} and P_{fa} (0.89±0.008 and 0.026±0.004, respectively), even though core temperature, and hence onset of febrile symptoms, is no longer an available feature. These results may be expected given the highly correlated nature of physiological data, but positively suggests the implementation of the present disclosure with non-invasive, ECG-based monitoring equipment. Specifically, even when all temperature, hemodynamic, and pulmonary features are excluded, the performance drops only slightly from Δtₘₑₐₙ 51 h to 46 h, and from pre-fever AUCROC=0.9515 to 0.9115. All other performance parameters are available in Table 6 below. These results indicate that this type of early warning algorithm may possibly be embedded on an ex vivo, wearable ECG system such as a Holter monitor.

The results shown in FIG. 15 suggest that the systems and methods of the present disclosure may include using signals from wearable sensing technologies. Electronics miniaturization has led to a wave of wearable sensing technologies for health monitoring, and increasingly more processing power is available to consumers to make meaningful use of these collected data. In particular, a low ergonomic profile, robust, wearable, personalized and multi-modal physiological monitoring system may persistently measure signals capable of sensitive pathogen exposure and infection detection. Such a system may cue the use of highly specific (but expensive) diagnostic tests, prompt low-regret responses such as patient isolation and observation, or advise clinicians of fulminant complications in already compromised patients.

Table 6 below includes system performance metrics for all validations. The aggregated three-fold cross-validation includes data from each of the three exposure studies in its training set. This same classifier is used to test independent LASV, NiV, and Y. pestis exposure study datasets including pre-exposure data from excluded subjects (see exclusion criteria under Description of Animal Studies subsection). The detection parameters for each study are m=11, n=24 and thresholds are estimated a priori for system P_{fa}=0.01. The broad distribution in Δt values both within and across pathogens can be understood both from the limited number of subjects for each pathogen (N_{LASV}=N_{y.pestis}=4 and N_{NiV}=5) and different lengths of each pathogens incubation and onset of prodromal periods.

TABLE 6

| | | | Pre- | Post- | | |
|---|---|---|---|---|---|---|
| | | Mean Δt ± | Fever | Fever | Pre-Fever P_{d} ± | System P_{d} & P_{fa} ± |
| Training Set | Test Set | 95% CI (h) | AUC | AUC | 95% CI | 95% CI |
| Aggregated from EBOV aerosol, MARV | | 51.0 ± 11.9 | 0.9343 | 0.9999 | 0.56 ± 0.02 | 0.80 ± 0.01 |
| aerosol, MARV IM studies | | | | | | 0.013 ± 0.003 |
| Aggregated | LASV | 32.6 ± 40.5 | 0.9515 | 0.9977 | 0.64 ± 0.05 | 0.94 ± 0.009 |
| EBOV and | | | | | | 0.040 ± 0.01 |
| MARV studies | NiV | 73.7 ± 37.2 | | | 0.46 ± 0.04 | 0.87 ± 0.01 |
| (above) | | | | | | 0.028 ± 0.01 |
| | Y. pestis | 40.8 ± 39.4 | | | 0.84 ± 0.04 | 0.90 ± 0.02 |
| | | | | | | 0.027 ± 0.01 |
| | All above pathogens | 51.0 ± 13.9 | | | 0.60 ± 0.03 | 0.90 ± .0007 |
| | plus pre-exposure | | | | | 0.025 ± 0.004 |
| | data from excluded | | | | | |
| | subjects | | | | | |
| Only ECG- | Only ECG-derived | 46.0 ± 14.1 | 0.9115 | 0.9978 | 0.55 ± 0.03 | 0.89 ± 0.008 |
| derived features | features from | | | | | 0.026 ± 0.004 |
| from Aggregated | independent datasets | | | | | |
| studies | | | | | | |

Experiment 1—Discussion

Non-biochemical detection of pathogen incubation periods using only physiological data presents an enabling new tool in infectious disease care. There is no existing method to detect non-symptomatic incubation period that is possibly extensible to mobile settings or wearable sensor systems, such as high-resolution ECG. The initial results described herein are presented towards building a multi-modal, supervised machine learning algorithm capable of determining this incubation period using only physiological waveforms, based on data collected in NHPs infected with several pathogens. Using the random forest method, over-fitting of the models is avoided, demonstrated by successful testing and training on both different subsets of data within the same exposure studies, as well as testing on entirely independent exposure datasets. These cross-validations show the promise of extending this approach beyond a given animal model, exposure method, or virus. While P_{fa}˜0.01 was selected for Experiment 1 (supported by the limited subject numbers in the studies available), this would not lead to an acceptable daily false alarm rate of about one declaration every 2 days (for 30 min windows). In some embodiments, P_{fa} may be be ˜10⁻³ or less, which corresponds to one false alarm approximately every 3 weeks of continuous monitoring (again, for 30 min windows). It may be possible to reduce this critical system parameter to more clinically acceptable levels if larger sample sizes are used, or more refined processing techniques are used. Furthermore, the effect of physiological confounders, such as intense exercise, arrhythmias, lifestyle diseases, and autochthonous or annual infections may be explored.

Immuno-biological events of the innate immune system—particularly systemic release of pro-inflammatory chemokines and cytokines from infected phagocytes, as well as afferent signaling to the central nervous system—may be recapitulated in hemodynamic, thermoregulatory, or cardiac signals which may be more easily measured and assessed than biomolecule markers for viral infection (via sequencing or immunocapture approaches). For instance, prostaglandins (PG) are up-regulated upon infection (including EBOV) and intricately involved in the non-specific “sickness syndrome”; the PGs are also known to be potent vascular mediators and endogenous pyrogens. Past work has clarified how tightly integrated, complex, and oscillating biological systems can become uncoupled during trauma or critical illness which would be captured in the comprehensive, multi-modal physiological datasets used in the present disclosure. Finding that the systems and methods of the present disclosure provide early warning times for both viral and (albeit limited) bacterial exposures suggests that the “exposure signal” found by the random forest models arises from the innate immune system, and is a generalized indication of immune activation rather than a specific signal for particular pathogens. Rigorously pursuing this hypothesis may involve additional high temporal resolution pathogen exposure datasets, including biochemical, immunological, neurological, and cardiovascular information. Transitioning this capability into clinical use may also involve the controlled exposure and monitoring of human subjects, such as during periodic influenza, tetanus, or zoster vaccinations.

Genomic profiles of peripheral blood cells following acute influenza infection indicate specific host responses at just ˜45 h following exposure, corresponding to ˜35 h of early warning time. The results of Experiment 1 described herein suggest that the classic understanding of a “non”-symptomatic incubation phase may be incomplete: during viral incubation, subtle sub-clinical cues (genomic, transcriptional, and physiological) can be detectable with sufficiently high-sensitivity sensor and analysis systems. Better understanding of how biomolecular changes are captured in systemic physiological signals during pathogen infection would open further opportunities for better therapeutic administration both before and during infection, quarantine or isolation, and vaccine development.

Detecting pathogen exposure before self-reporting or overt clinical symptoms affords great opportunities in clinical care and public health measures. However, given the consequences of using some of these interventions and the lack of etiological agent specificity in the present disclosure, this current approach (after appropriate human testing) may be a trigger for ‘low-regret’ actions rather than necessarily guiding medical care. For instance, using the high sensitivity approach described herein as an alert for limited high specificity confirmatory diagnostics, such as sequencing or PCR-based, may lead to considerable cost savings (an “alert-confirm” system). Public health response following a bioterrorism incident may also benefit from triaging those exposed from the “worried well.” It may be desirable to add enough causative agent specificity to discern between bacterial and viral pathogens. Even this binary classification would be of use for front-line therapeutic or mass casualty uses. The systems and methods of the present disclosure may provide real-time prognostic information, even before obvious illness, guiding patients and clinicians in diagnostic or therapeutic use with better time resolution than ever before.

Implementing a type of early-warning system, as disclosed herein, could save lives of health care workers, military service members, patients, and other susceptible individuals. During the 2014 West Africa Ebola outbreak, for instance, health care workers at higher risk of viral exposure could have been monitored persistently for the earliest possible indications of viral exposure. More commonly, patients in post-operative or critical care units could be monitored for infection and treated well before clinical symptoms, viremia/bacteremia, or septic shock. Higher specificity iterations of this approach and knowledge of the causative agent could inform very early therapeutic intervention without departing from the scope of the disclosure. Furthermore, using very feature sparse datasets, such as those that could be collected using wearable sensor platforms, would enable this technique to be implemented in, for example, rugged military environments.

While various embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A method for predicting whether a patient has been exposed to an agent, the method comprising, for each respective time interval in a plurality of time intervals:
(a) receiving, by at least one processor, physiological data regarding the patient that was recorded during the respective time interval;
(b) extracting one or more features from the physiological data, wherein each feature is representative of the physiological data during the respective time interval;
(c) for each respective classifier in a plurality of classifiers:
(i) identifying the respective classifier, wherein the respective classifier is trained using training data for a respective physiological state;
(ii) applying the respective classifier to the one or more features to obtain a classifier output that represents a likelihood that the patient has been exposed to the agent;
(iii) applying a respective first threshold to the classifier output to determine a patient state classification;
(iv) aggregating the patient state classifications across a number of time intervals to obtain an aggregate patient state classification for each classifier;
(d) combining the aggregate patient state classifications across the plurality of classifiers to obtain a combined classification; and
(e) providing an indication that the patient has been exposed to the agent when the combined classification exceeds a second threshold.

2. The method of claim 1, wherein:
the plurality of classifiers includes a first classifier and a second classifier;
the first classifier is trained using pre-fever training data; and
the second classifier is trained using post-fever training data.

3. The method of claim 2, wherein the plurality of classifiers further includes a third classifier that is trained using training data following the pre-fever training data and preceding the post-fever training data.

4. The method of claim 1, wherein each extracted feature in (b) is further representative of the physiological data during at least one time interval previous to the respective time interval.

5. The method of claim 1, wherein the respective first thresholds at (c)(iii) are determined based on a desired probability of false alarm for each respective classifier.

6. The method of claim 1, wherein the second threshold is determined based on a performance metric of the system that is related to a probability of false alarm, a probability of detection, or early warning purity.

7. The method of claim 1, wherein the patient state classification in (c)(iii) is a binary value indicative of a prediction by the respective classifier of whether the patient is exposed or not exposed, and the aggregating in (c)(iv) includes summing across the binary values.

8. The method of claim 7, wherein the aggregating in (c)(iv) further includes normalizing the summed binary values by the number of time intervals to obtain an averaged score for each respective classifier.

9. The method of claim 8, wherein the combining in (d) includes determining a maximum averaged score across the plurality of classifiers.

10. The method of claim 9, wherein the second threshold in (e) is determined based on a ratio m/n, where n is the number of time intervals in (c)(iv) and m is an integer greater than 0 and less than or equal to n.

11. The method of claim 1, wherein the physiological data solely includes an electrocardiogram signal obtained from a non-invasive wearable device on the patient.

12. The method of claim 1, wherein the physiological data solely includes an electrocardiogram signal and a temperature signal obtained from at least one non-invasive wearable device on the patient.

13. The method of claim 1, wherein the one or more features include solely heart rate and temperature.

14. The method of claim 1, wherein the agent is a first agent, and the training data includes data from subjects that were exposed to a second agent that is different from the first agent.

15. The method of claim 1, wherein the patient is a human, and the training data includes data from non-human animal subjects.

16. The method of claim 1, wherein the extracting in (b) includes standardizing the physiological data such that the extracted one or more features are allowed to be compared across the respective time intervals.

17. A system for predicting whether a patient has been exposed to an agent, the system comprising at least one processor configured to, for each respective time interval in a plurality of time intervals:
(a) receive physiological data regarding the patient that was recorded during the respective time interval;
(b) extract one or more features from the physiological data, wherein each feature is representative of the physiological data during the respective time interval;
(c) for each respective classifier in a plurality of classifiers:
(i) identify the respective classifier, wherein the respective classifier is trained using training data for a respective physiological state;
(ii) apply the respective classifier to the one or more features to obtain a classifier output that represents a likelihood that the patient has been exposed to the agent;
(iii) apply a respective first threshold to the classifier output to determine a patient state classification;
(iv) aggregate the patient state classifications across a number of time intervals to obtain an aggregate patient state classification for each classifier;
(d) combine the aggregate patient state classifications across the plurality of classifiers to obtain a combined classification; and
(e) provide an indication that the patient has been exposed to the agent when the combined classification exceeds a second threshold.

18. The system of claim 17, wherein:
the plurality of classifiers includes a first classifier and a second classifier;
the first classifier is trained using pre-fever training data; and
the second classifier is trained using post-fever training data.

19. The system of claim 18, wherein the plurality of classifiers further includes a third classifier that is trained using training data following the pre-fever training data and preceding the post-fever training data.

20. The system of claim 17, wherein the physiological data solely includes an electrocardiogram signal obtained from a non-invasive wearable device on the patient.

21. The system of claim 17, wherein the physiological data solely includes an electrocardiogram signal and a temperature signal obtained from at least one non-invasive wearable device on the patient.

22. The system of claim 17, wherein the one or more features include solely heart rate and temperature.

23. The system of claim 17, wherein the agent is a first agent, and the training data includes data from subjects that were exposed to a second agent that is different from the first agent.

24. The system of claim 17, wherein the patient is a human, and the training data includes data from non-human animal subjects.
